(19) United States
(12) Patent Application Publication
(10) Pub. No.: US 2004/0223911 A1
(43) Pub. Date: Nov. 11, 2004

(54) Stabilized therapeutic and imaging agents

(76) Inventors: Mark David Bednarski, Los Altos, CA (US); Neal Edward DeChene, Morgan Hill, CA (US); John S. Pease, Los Altos, CA (US); Charles Aaron Wartchow, San Francisco, CA (US); Karen J. Brunke, Belmont, CA (US)

Correspondence Address: SWANSON & BRATSCHUN L.L.C., 1745 SHEA CENTER DRIVE, SUITE 330, HIGHLANDS RANCH, CO, 80129, US

(21) Appl. No.: 10/863,734

(22) Filed: Jun. 8, 2004

Related U.S. Application Data
(63) Continuation of application No. 10/093,845, filed on Mar. 8, 2002, now abandoned.
(60) Provisional application No. 60/274,361, filed on Mar. 8, 2001.

(51) Int. Cl. A61K 51/00; A61K 9/127

(52) U.S. Cl. 424/1.21; 424/450

(57) ABSTRACT

Stabilized lipid construct comprising a liposome or polymerized vesicle, a targeting entity, a therapeutic entity, and a stabilizing entity are provided, as well as methods for their preparation and use.

### CROSS-REFERENCE TO RELATED APPLICATIONS

This application is a continuation of U.S. application Ser. No. 10/093,845, filed Mar. 8, 2002, incorporated by reference herein in its entirety, which claims priority under 35 U.S.C. § 119 from U.S. application Ser. No. 60/274,361, filed Mar. 8, 2001.

### FIELD OF THE INVENTION

This invention relates to therapeutic and imaging agents which are comprised of a targeting entity, a therapeutic or treatment entity and a linking carrier. In preferred agents of the present invention comprise a lipid construct, vesicle, liposome, or polymerized liposome. The therapeutic or treatment entity may be associated with the agent by covalent or non-covalent means. In some cases, the therapeutic or treatment entity is a radioisotope, chemotherapeutic agent, prodrug, toxin, or gene encoding a protein that exhibits cell toxicity. Preferably, the agent is further comprised of a stabilizing entity that imparts additional advantages to the therapeutic or imaging agent. The stabilizing entity may be associated with the agent by covalent or non-covalent means. Preferably, the stabilizing entity is dextran, which preferably forms a coating on the surface of the lipid construct, vesicle, liposome, or polymerized liposome. In preferred embodiments the linking carrier is a polymerized liposome. The linking carrier imparts additional advantages to the therapeutic agents, which are not provided by conventional linking methods.

### BACKGROUND OF THE INVENTION

Cancer remains one of the leading causes of death in the industrialized world. In the United States, cancer is the second most common cause of death after heart disease, accounting for approximately one-quarter of the deaths in 1997. Clearly, new and effective treatments for cancer will provide significant health benefits. Among the wide variety of treatments proposed for cancer, targeted therapeutic agents hold considerable promise. In principle, a patient could tolerate much higher doses of a cytotoxic agent if the cytotoxic agent is targeted specifically to cancerous tissue, as healthy tissue should be unaffected or affected to a much smaller extent than the pathological tissue.

Due to the high specificity of monoclonal antibodies, antibodies coupled to cytotoxic agents have been proposed for targeted cancer treatment therapies. Solid tumors, in particular, express certain antigens, on both the transformed cells comprising the tumor and the vasculature supplying the tumors, which are either unique to the tumor cells and vasculature, or overexpressed in tumor cells and vasculature in comparison to normal cells and vasculature. Thus, linking an antibody specific for a tumor antigen, or a tumor vasculature antigen, to a cytotoxic agent, should provide high specificity to the site of pathology. One group of such antigens is a family of proteins called cell adhesion molecules (CAMS), expressed by endothelial cells during a variety of physiological and disease processes. Reisfeld, “Monoclonal Antibodies in Cancer Immunotherapy,” Laboratory Immunology II, (1992) 12(2):201-216, and Archelos et al., “Inhibition of Experimental Autoimmune Encephalomyelitis by the Antibody to the Intercellular Adhesion Molecule ICAM-1,” Ann. of Neurology (1993) 34(2):145-154. Multiple endothelial ligands and receptors, including CAMs, are known to be upregulated during various pathologies, such as inflammation and neoplasia, and hence are attractive candidates for targeting strategies.

Other potential targets are integrins. Integrins are a group of cell surface glycoproteins that mediate cell adhesion and therefore are mediators of cell adhesion interactions that occur in various biological processes. Integrins are heterodimers composed of noncovalently linked α and β polypeptide subunits. Currently at least eleven different a subunits have been identified and at least six different β subunits have been identified. The various α subunits can combine with various β subunits to form distinct integrins. The integrin identified as αᵥβ₃ (also known as the vitronectin receptor) has been identified as an integrin that plays a role in various conditions or disease states including but not limited to tumor metastasis, solid tumor growth (neoplasia), osteoporosis, Paget's disease, humoral hypercalcemia of malignancy, angiogenesis, including tumor angiogenesis, retinopathy, macular degeneration, arthritis, including rheumatoid arthritis, periodontal disease, psoriasis and smooth muscle cell migration (e.g., restenosis). Additionally, it has been found that such integrin inhibiting agents would be useful as antivirals, antifungals and antimicrobials. Thus, therapeutic agents that selectively inhibit or antagonize αᵥβ₃ would be beneficial for treating such conditions. It has been shown that the αᵥβ₃ integrin binds to a number of Arg-Gly-Asp (RGD) containing matrix macromolecules, such as fibrinogen (Bennett et al., Proc. Natl. Acad. Sci. USA, Vol. 80 (1983) 2417), fibronectin (Ginsberg et al., J. Clin. Invest., Vol. 71 (1983) 619-624), and von Willebrand factor (Ruggeri et al., Proc. Natl. Acad. Sci. USA, Vol. 79 (1982) 6038). Compounds containing the RGD sequence mimic extracellular matrix ligands so as to bind to cell surface receptors. However, it is also known that RGD peptides in general are non-selective for RGD dependent integrins. For example, most RGD peptides that bind to αᵥβ₃ also bind to αᵥβ₅, αᵥβ₁, and α_{IIb}β_{IIIa}. Antagonism of platelet α_{IIb}β_{IIIa} (also known as the fibrinogen receptor) is known to block platelet aggregation in humans.

A number of anti-integrin antibodies are known. Doerr, et al., J. Biol. Chem. 1996 271:2443 reported that a blocking antibody to αᵥβ₅ integrin in vitro inhibits the migration of MCF-7 human breast cancer cells in response to stimulation from IGF-1. Gui et al., British J. Surgery 1995 82:1192, report that antibodies against αᵥβ₅ and αᵥβ₁ inhibit in vitro chemoinvasion by human breast cancer carcinoma cell lines Hs578T and MDA-MB-231. Lehman et al., Cancer Research 1994 54:2102 show that a monoclonal antibody (69-6-5) reacts with several αᵥ integrins including αᵥβ₃ and inhibited colon carcinoma cell adhesion to a number of substrates, including vitronectin. Brooks et al., Science 1994 264:569 show that blockade of integrin activity with an anti-αᵥβ₃ monoclonal antibody inhibits tumor-induced angiogenesis of chick chorioallantoic membranes by human M21-L melanoma fragments. Chuntharapai, et al., Exp. Cell. Res. 1993 205:345 discloses monoclonal antibodies 9G2.1.3 and IOC4.1.3 which recognize the αᵥβ₃ complex, the latter monoclonal antibody is said to bind weakly or not at all to tissues expressing αᵥβ₃ with the exception of osteoclasts and was suggested to be useful for in vivo therapy of bone disease. The former monoclonal antibody is suggested to have potential as a therapeutic agent in some cancers.

Ginsberg et al., U.S. Pat. No. 5,306,620 discloses antibodies that react with integrin so that the binding affinity of integrin for ligands is increased. As such these monoclonal antibodies are said to be useful for preventing metastasis by immobilizing melanoma tumors. Brown, U.S. Pat. No. 5,057,604 discloses the use of monoclonal antibodies to αᵥβ₃ integrins that inhibit RGD-mediated phagocytosis enhancement by binding to a receptor that recognizes RGD sequence containing proteins. Plow et al., U.S. Pat. No. 5,149,780 discloses a protein homologous to the RGD epitope of integrin β subunits and a monoclonal antibody that inhibits integrin-ligand binding by binding to the β₃ subunit. That action is said to be of use in therapies for adhesion-initiated human responses such as coagulation and some inflammatory responses.

Carron, U.S. Pat. No. 6,171,588, describes monoclonal antibodies which can be used in a method for blocking αᵥβ₃-mediated events such as cell adhesion, osteoclast-mediated bone resorption, restenosis, ocular neovascularization and growth of hemangiomas, as well as neoplastic cell or tumor growth and dissemination. Other uses described are antibody-mediated targeting and delivery of therapeutics for disrupting or killing αᵥβ₃ bearing neoplasms and tumor-related vascular beds. In addition, the inventive monoclonal antibodies can be used for visualization or imaging of αᵥβ₃-bearing neoplasms or tumor-related vascular beds by NMR or immunoscintigraphy.

Examples of the targeted therapeutic approach have been described in various patent publications and scientific articles. International Patent Application WO 93/17715 describes antibodies carrying diagnostic or therapeutic agents targeted to the vasculature of solid tumor masses through recognition of tumor vasculature-associated antigens. International Patent Application WO 96/01653 and U.S. Pat. No. 5,877,289 describe methods and compositions for in vivo coagulation of tumor vasculature through the site-specific delivery of a coagulant using an antibody, while International Patent Application WO 98/31394 describes use of Tissue Factor compositions for coagulation and tumor treatment. International Patent Application WO 93/18793 and U.S. Pat. Nos. 5,762,918 and 5,474,765 describe steroids linked to polyanionic polymers which bind to vascular endothelial cells. International Patent Application WO 91/07941 and U.S. Pat. No. 5,165,923 describe toxins, such as ricin A, bound to antibodies against tumor cells. U.S. Pat. Nos. 5,660,827, 5,776,427, 5,855,866, and 5,863,538 also disclose methods of treating tumor vasculature. International Patent Application WO 98/10795 and WO 99/13329 describe tumor homing molecules, which can be used to target drugs to tumors.

In Tabata, et al., Int. J. Cancer 1999 82:737-42, antibodies are used to deliver radioactive isotopes to proliferating blood vessels. Ruoslahti & Rajotte, Annu. Rev. Immunol. 2000 18:813-27; Ruoslahti, Adv. Cancer Res. 1999 76:1-20, review strategies for targeting therapeutic agents to angiogenic neovasculature, while Arap, et al., Science 1998 279:377-80 describe selection of peptides which target tumor blood vessels.

It should be noted that the typical arrangement used in such systems is to link the targeting entity to the therapeutic entity via a single bond or a relatively short chemical linker. Examples of such linkers include SMCC (succinimidyl 4-[N-maleimidomethyl]cyclohexane-1-carboxylate) or the linkers disclosed in U.S. Pat. No. 4,880,935, and oligopeptide spacers. Carbodiimides and N-hydroxysuccinimide reagents have been used to directly join therapeutic and targeting entities with the appropriate reactive chemical groups.

The use of cationic organic molecules to deliver heterologous genes in gene therapy procedures has been reported in the literature. Not all cationic compounds will complex with DNA and facilitate gene transfer. Currently, a primary strategy is routine screening of cationic molecules. The types of compounds which have been used in the past include cationic polymers such as polyethyleneamine, ethylene diamine cascade polymers, and polybrene. Proteins, such as polylysine with a net positive charge, have also been used. The largest group of compounds, cationic lipids; includes DOTMA, DOTAP, DMRIE, DC-chol, and DOSPA. All of these agents have proven effective but suffer from potential problems such as toxicity and expense in the production of the agents. Cationic liposomes are currently the most popular system for gene transfection studies. Cationic liposomes serve two functions: protect DNA from degradation and increase the amount of DNA entering the cell. While the mechanisms describing how cationic liposomes function have not been fully delineated, such liposomes have proven useful in both in vitro and in vivo studies. However, these liposomes suffer from several important limitations. Such limitations include low transfection efficiencies, expense in production of the lipids, poor colloidal stability when complexed to DNA, and toxicity.

Although conjugates of targeting entities with therapeutic entities via relatively small linkers have attracted much attention, far less attention has been focused on using large particles as linkers. Typically, the linker functions simply to connect the therapeutic and targeting entities, and consideration of linker properties generally focuses on avoiding interference with the entities linked, for example, avoiding a linkage point in the antigen binding site of an immunoglobulin.

Large particulate assemblies of biologically compatible materials, such as liposomes, have been used as carriers for administration of drugs and paramagnetic contrast agents. U.S. Pat. Nos. 5,077,057 and 5,277,914 teach preparation of liposome or lipidic particle suspensions having particles of a defined size, particularly lipids soluble in an aprotic solvent, for delivery of drugs having poor aqueous solubility. U.S. Pat. No. 4,544,545 teaches phospholipid liposomes having an outer layer including a modified, cholesterol derivative to render the liposome more specific for a preselected organ. U.S. Pat. No. 5,213,804 teaches liposome compositions containing an entrapped agent, such as a drug, which are composed of vesicle-forming lipids and 1 to 20 mole percent of a vesicle-forming lipid derivatized with hydrophilic biocompatible polymer and sized to control its biodistribution and recirculatory half life. U.S. Pat. No. 5,246,707 teaches phospholipid-coated microcrystalline particles of bioactive material to control the rate of release of entrapped water-soluble biomolecules, such as proteins and polypeptides. U.S. Pat. No. 5,158,760 teaches liposome encapsulated radioactive labeled proteins, such as hemoglobin.

U.S. Pat. Nos. 5,512,294 and 6,090,408, and 6,132,764 describe the use of polymerized liposomes for various biological applications. The contents of these patents, and all others patents and publications referred to herein, are incorporated by reference herein in their entireties. One listed embodiment is to targeted polymerized liposomes which may be linked to or may encapsulate a therapeutic compound (e.g. proteins, hormones or drugs), for directed delivery of a treatment agent to specific biological locations for localized treatment. Other publications describing liposomal compositions include U.S. Pat. Nos. 5,663,387, 5,494,803, and 5,466,467. Liposomes containing polymerized lipids for non-covalent immobilization of proteins and enzymes are described in Storrs et al., “Paramagnetic Polymerized Liposomes: Synthesis, Characterization, and Applications for Magnetic Resonance Imaging,” J. Am. Chem. Soc. (1995) 117(28):7301-7306; and Storrs et al., “Paramagnetic Polymerized Liposomes as New Recirculating MR Contrast Agents,” JMRI (1995) 5(6):719-724. Wu et al., “Metal-Chelate-Dendrimer-Anntibody Constructs for Use in Radioimmunotherapy and Imaging,” Bioorganic and Medicinal Chemistry Letters (1994) 4(3):449-454, is a publication directed to dendrimer-based compounds.

The need for recirculation of therapeutic agents in the body, that is avoidance of rapid endocytosis by the reticuloendothelial system and avoidance of rapid filtration by the kidney, to provide sufficient concentration at a targeted site to afford necessary therapeutic effect has been recognized. Experience with magnetic resonance contrast agents has provided useful information regarding circulation lifetimes. Small molecules, such as gadolinium diethylenetriaminepentaacetic acid, tend to have limited circulation times due to rapid renal excretion while most liposomes, having diameters greater than 800 nm, are quickly cleared by the reticuloendothelial system. Attempts to solve these problems have involved use of macromolecular materials, such as gadolinium diethylenetriaminepentaacetic acid-derived polysaccharides, polypeptides, and proteins. These agents have not achieved the versatility in chemical modification to provide for both long recirculation times and active targeting.

Stabilization

The association of liposomes with polymeric compounds in order to avoid rapid clearance in the liver, or for other stabilizing effects, has been described. For example, Dadey, U.S. Pat. No. 5,935,599 described polymer-associated liposomes containing a liposome, and a polymer having a plurality of anionic moieties in a salt form. The polymer may be synthetic or naturally-occurring. The polymer-associated liposomes remain in the vascular system for an extended period of time.

Polysaccharides are one class of polymeric stabilizer. Calvo Salve, et al., U.S. Pat. No. 5,843,509 describe the stabilization of colloidal systems through the formation of lipid-polysaccharide complexes and development of a procedure for the preparation of colloidal systems involving a combination of two ingredients: a water soluble and positively charged polysaccharide and a negatively-charged phospholipid. Stabilization occurs through the formation, at the interface, of an ionic complex: aminopolysaccharide-phospholipid. The polysaccharides utilized by Calvo Salve, et al., include chitin and chitosan.

Dextran is another polysaccharide whose stabilizing properties have been investigated. Cansell, et al., J. Biomed. Mater. Res. 1999, 44:140-48, report that dextran or functionalized dextran was hydrophobized with cholesterol, which anchors in the lipid bilayer of liposomes during liposome formation, resulting in a liposome coated with dextran. These liposomes interacted specifically with human endothelial cells in culture. In Letourneur, et al., J. Controlled Release 2000, 65:83-91, the antiproliferative functionalized dextran-coated liposomes were used as a targeting agent for vascular smooth muscle cells. Ullman, et al. Proc. Nat. Acad. Sci 91:5426-30 (1994) and Ullman, et al., Clin. Chem. 42:1518-26 (1996) describe the coating of polystyrene beads with dextran and the attachment of ligands, nucleic acids, and proteins to the dextran-polystyrene complexes.

Dextran has also been used to coat metal nanoparticles, and such nanoparticles have been used primarily as imaging agents. For example, Moore, et al., Radiology 2000, 214:568-74, report that in a rodent model, long-circulating dextran-coated iron oxide nanoparticles were taken up preferentially by tumor cells, but also were taken up by tumor-associated macrophages and, to a much lesser extent, endothelial cells in the area of angiogenesis. Groman, et al., U.S. Pat. No. 4,770,183, describe 10-5000 Å superparamagnetic metal oxide particles for use as imaging agents. The particles may be coated with dextran or other suitable polymer to optimize both the uptake of the particles and the residence time in the target organ. A dextran-coated iron oxide particle injected into a patient's bloodstream, for example, localizes in the liver. Groman, et al., also report that dextran-coated particles can be preferentially absorbed by healthy cells, with less uptake into cancerous cells.

Imaging

Magnetic resonance imaging (MRI) is an imaging technique which, unlike X-rays, does not involve ionizing radiation. MRI may be used for producing cross-sectional images of the body in a variety of scanning planes such as, for example, axial, coronal, sagittal or orthogonal. MRI employs a magnetic field, radio-frequency energy and magnetic field gradients to make images of the body. The contrast or signal intensity differences between tissues mainly reflect the T1 (longitudinal) and T2 (transverse) relaxation values and the proton density in the tissues. To change the signal intensity in a region of a patient by the use of a contrast medium, several possible approaches are available. For example, a contrast medium may be designed to change either the T1, the T2 or the proton density.

Generally speaking, MRI requires the use of contrast agents. If MRI is performed without employing a contrast agent, differentiation of the tissue of interest from the surrounding tissues in the resulting image may be difficult. In the past, attention has focused primarily on paramagnetic contrast agents for MRI. Paramagnetic contrast agents involve materials which contain unpaired electrons. The unpaired electrons act as small magnets within the main magnetic field to increase the rate of longitudinal (T1) and transverse (T2) relaxation. Paramagnetic contrast agents typically comprise metal ions, for example, transition metal ions, which provide a source of unpaired electrons. However, these metal ions are also generally highly toxic. For example, ferrites often cause symptoms of nausea after oral administration, as well as flatulence and a transient rise in serum iron. The gadolinium ion, which is complexed in Gd-DTPA, is highly toxic in free form. The various environments of the gastrointestinal tract, including increased acidity (lower pH) in the stomach and increased alkalinity (higher pH) in the intestines, may increase the likelihood of decoupling and separation of the free ion from the complex. In an effort to decrease toxicity, the metal ions are typically chelated with ligands.

Ultrasound is another valuable diagnostic imaging technique for studying various areas of the body, including, for example, the vasculature, such as tissue microvasculature. Ultrasound provides certain advantages over other diagnostic techniques. For example, diagnostic techniques involving nuclear medicine and X-rays generally involve exposure of the patient to ionizing electron radiation. Such radiation can cause damage to subcellular material, including deoxyribonucleic acid (DNA), ribonucleic acid (RNA) and proteins. Ultrasound does not involve such potentially damaging radiation. In addition, ultrasound is inexpensive relative to other diagnostic techniques, including CT and MRI, which require elaborate and expensive equipment.

Ultrasound involves the exposure of a patient to sound waves. Generally, the sound waves dissipate due to absorption by body tissue, penetrate through the tissue or reflect off of the tissue. The reflection of sound waves off of tissue, generally referred to as backscatter or reflectivity, forms the basis for developing an ultrasound image. In this connection, sound waves reflect differentially from different body tissues. This differential reflection is due to various factors, including the constituents and the density of the particular tissue being observed. Ultrasound involves the detection of the differentially reflected waves, generally with a transducer that can detect sound waves having a frequency of one to ten megahertz (MHz). The detected waves can be integrated into an image which is quantitated and the quantitated waves converted into an image of the tissue being studied.

As with the diagnostic techniques discussed above, ultrasound also generally involves the use of contrast agents. Exemplary contrast agents include, for example, suspensions of solid particles, emulsified liquid droplets, and gas-filled bubbles (see, e.g., Hilmann et al., U.S. Pat. No. 4,466,442, and published International Patent Applications WO 92/17212 and WO 92/21382). Widder et al., published application EP-A-0 324 938, disclose stabilized microbubble-type ultrasonic imaging agents produced from heat-denaturable biocompatible protein, for example, albumin, hemoglobin, and collagen.

The reflection of sound from a liquid-gas interface is extremely efficient. Accordingly, liposomes or vesicles, including gas-filled bubbles, are useful as contrast agents. As discussed more fully hereinafter, the effectiveness of liposomes as contrast agents depends upon various factors, including, for example, the size and/or elasticity of the bubble.

Many of the liposomes disclosed in the prior art have undesirably poor stability. Thus, the prior art liposomes are more likely to rupture in vivo resulting, for example, in the untimely release of any therapeutic and/or diagnostic agent contained therein. Various studies have been conducted in an attempt to improve liposome stability. Such studies have included, for example, the preparation of liposomes in which the membranes or walls thereof comprise proteins, such as albumin, or materials which are apparently strengthened via crosslinking. See, e.g., Klaveness et al., WO 92/17212, in which there are disclosed liposomes which comprise proteins crosslinked with biodegradable crosslinking agents. A presentation was made by Moseley et al., at a 1991 Napa, California meeting of the Society for Magnetic Resonance in Medicine, which is summarized in an abstract entitled “Microbubbles: A Novel MR Susceptibility Contrast Agent.” The microbubbles described by Moseley et al. comprise air coated with a shell of human albumin. Alternatively, membranes can comprise compounds which are not proteins but which are crosslinked with biocompatible compounds. See, e.g., Klaveness et al., WO 92/17436, WO 93/17718 and WO 92/21382.

Prior art techniques for stabilizing liposomes, including the use of proteins in the outer membrane, suffer from various drawbacks. The use in membranes of proteins, such as albumin, can impart rigidity to the walls of the bubbles. This results in bubbles having educed elasticity and, therefore, a decreased ability to deform and pass through capillaries. Thus, there is a greater likelihood of occlusion of vessels with prior art contrast agents that involve proteins.

### SUMMARY OF THE INVENTION

This invention relates to therapeutic and imaging agents which are comprised of a targeting entity, a therapeutic or treatment entity and a linking carrier. Preferred agents of the present invention are comprised of a lipid construct, vesicle, liposome, or polymerized liposome. The therapeutic or treatment entity may be associated with the linking carrier by covalent or non-covalent means. In some cases, the therapeutic or treatment entity is a radioisotope, chemotherapeutic agent, prodrug, or toxin. Preferably, the agent is further comprised of a stabilizing entity which imparts additional advantages to the therapeutic or imaging agent. The stabilizing entity may be associated with the agent by covalent or non-covalent means. Preferably, the stabilizing entity is dextran, which preferably forms a coating on the surface of the agent by covalent or non-covalent means. In the most preferred embodiments, the linking carrier is a vesicle. The linking carrier imparts additional advantages to the therapeutic agents, which are not provided by conventional linking methods.

The present invention is also directed toward vascular-targeted imaging agents comprised of a targeting entity, an imaging entity, a stabilizing entity, and optionally, a linking carrier. The present invention is further directed toward diagnostic agents comprised of a targeting entity, a detection entity, a stabilizing entity, and optionally, a linking carrier.

The present invention is also directed toward methods for preparing the aforementioned therapeutic and imaging agents.

The present invention is also directed toward therapeutic compositions comprising the therapeutic agents of the present invention.

The present invention is also directed toward methods of treatment utilizing the therapeutic agents of the present invention.

The present invention is also directed toward compositions for imaging comprising imaging agents of the present invention.

The present invention is also directed toward methods for utilizing the imaging agents of the present invention, including a method for diagnosing cancer.

The present invention is also directed toward methods and reagents for use in diagnostic assays.

### BRIEF DESCRIPTION OF THE DRAWINGS

FIG. 1A-D shows schematics of an exemplary lipid construct of the present invention.

FIG. 2 shows lipids used for the preparation of stabilized lipid constructs of the invention.

FIG. 3 shows mean vesicle diameter vs. vesicle type for polymerized vesicles in the presence and absence of 200 mM NaCl.

FIG. 4 shows a comparison of in vitro delivery of yttrium-90 for therapeutic stabilized and unstabilized polymerized vesicles in rabbit serum.

FIG. 5 shows a comparison of stability of therapeutic stabilized and unstabilized polymerized vesicles in rabbit serum.

FIG. 6 shows the result of treatment of melanoma in a murine tumor model with anti-VEGFR2 antibody (Ab), anti-VEGFR2 Ab-dextran-polymerized vesicle conjugates (anti-VEGFR2-dexPV), dextran-polymerized vesicle-yttrium-90 complexes (dexPV-Y90), and anti-VEGFR2 Ab-dextran-polymerized vesicle-yttrium-90 complexes (anti-VEGFR2-dexPV-Y90).

FIG. 7 shows a comparison of the effect of various of antibody-dextran-polymerized vesicle-yttrium-90 conjugates in the murine melanoma tumor model.

### DETAILED DESCRIPTION OF THE PREFERRED EMBODIMENTS

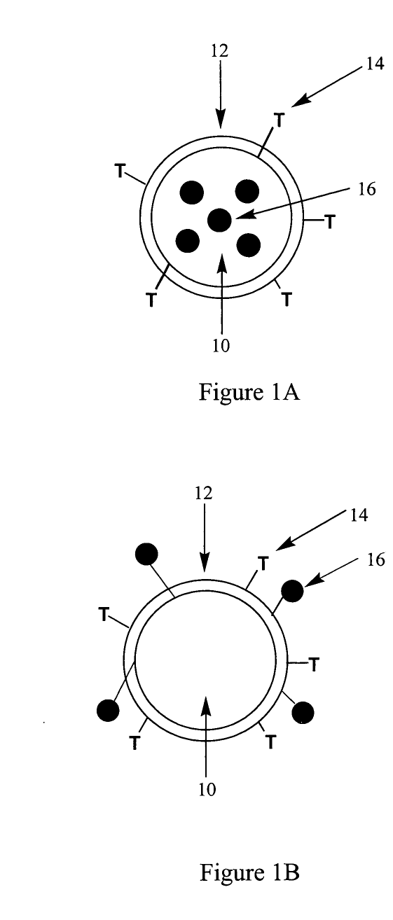

This invention relates to stabilized therapeutic and imaging agents, examples of which are shown schematically in FIG. 1A, 1B, 1C, and 1D, which are comprised of a lipid construct, 10, a stabilizing agent, 12, a targeting entity 14, and/or a therapeutic or treatment entity, 16. As depicted in FIG. 1A and 1B, the targeting and/or therapeutic entities may be associated with the lipid construct or the stabilizing entity. FIGS. 1A, 1B, 1C, and 1D show examples comprise both a therapeutic or targeting agent, but the agents of the invention may contain a therapeutic entity, a targeting entity, or both. Additionally, the therapeutic entity may be encapsulated within the lipid construct, or may be associated with the surface of the lipid construct or stabilizing agent.

A “lipid construct,” as used herein, is a structure containing lipids, phospholipids, or derivatives thereof comprising a variety of different structural arrangements which lipids are known to adopt in aqueous suspension. These structures include, but are not limited to, lipid bilayer vesicles, micelles, liposomes, emulsions, lipid ribbons or sheets, and may be complexed with a variety of drugs and components which are known to be pharmaceutically acceptable. In the preferred embodiment, the lipid construct is a liposome. Common adjuvants include cholesterol and alpha-tocopherol, among others. The lipid constructs may be used alone or in any combination which one skilled in the art would appreciate to provide the characteristics desired for a particular application. In addition, the technical aspects of lipid construct, vesicle, and liposome formation are well known in the art and any of the methods commonly practiced in the field may be used for the present invention. The therapeutic or treatment entity may be associated with the agent by covalent or non-covalent means. Preferably, the agent is further comprised of a stabilizing entity which imparts additional advantages to the therapeutic or imaging agent which are not provided by conventional stabilizing entities. The stabilizing entity may be associated with the agent by covalent or non-covalent means. As used herein, associated means attached to by covalent or noncovalent interactions. Once the stabilizing entity is associated with the agent, the agent may be referred to as a “stabilized agent,” or in a more specific fashion depending on the type of lipid construct used, i.e., “stabilized liposome,” or “stabilized polymerized liposome.”

Therapeutic Entities

The term “therapeutic entity” refers to any molecule, molecular assembly or macromolecule that has a therapeutic effect in a treated subject, where the treated subject is an animal, preferably a mammal, more preferably a human. The term “therapeutic effect” refers to an effect which reverses a disease state, arrests a disease state, slows the progression of a disease state, ameliorates a disease state, relieves symptoms of a disease state, or has other beneficial consequences for the treated subject. Therapeutic entities include, but are not limited to, drugs, such as doxorubicin and other chemotherapy agents; small molecule therapeutic drugs, toxins such as ricin; radioactive isotopes; genes encoding proteins that exhibit cell toxicity, and prodrugs (drugs which are introduced into the body in inactive form and which are activated in situ). Radioisotopes useful as therapeutic entities are described in Kairemo, et al., Acta Oncol. 35:343-55 (1996), and include Y-90, I-123, I-125, I-131, Bi-213, At-211, Cu-67, Sc-47, Ga-67, Rh-105, Pr-142, Nd-147, Pm-151, Sm-153, Ho-166, Gd-159, Th-161, Eu-152, Er-171, Re-186, and Re-188.

Liposomes

As used herein, lipid refers to an agent exhibiting amphipathic characteristics causing it to spontaneously adopt an organized structure in water wherein the hydrophobic portion of the molecule is sequestered away from the aqueous phase. A lipid in the sense of this invention is any substance with characteristics similar to those of fats or fatty materials. As a rule, molecules of this type possess an extended apolar region and, in the majority of cases, also a water-soluble, polar, hydrophilic group, the so-called head-group. Phospholipids are lipids which are the primary constituents of cell membranes. Typical phospholipid hydrophilic groups include phosphatidylcholine and phosphatidylethanolamine moieties, while typical hydrophobic groups include a variety of saturated and unsaturated fatty acid moieties, including diacetylenes. Mixture of a phospholipid in water causes spontaneous organization of the phospholipid molecules into a variety of characteristic phases depending on the conditions used. These include bilayer structures in which the hydrophilic groups of the phospholipids interact at the exterior of the bilayer with water, while the hydrophobic groups interact with similar groups on adjacent molecules in the interior of the bilayer. Such bilayer structures can be quite stable and form the principal basis for cell membranes.

Bilayer structures can also be formed into closed spherical shell-like structures which are called vesicles or liposomes. The liposomes employed in the present invention can be prepared using any one of a variety of conventional liposome preparatory techniques. As will be readily apparent to those skilled in the art, such conventional techniques include sonication, chelate dialysis, homogenization, solvent infusion coupled with extrusion, freeze-thaw extrusion, microemulsification, as well as others. These techniques, as well as others, are discussed, for example, in U.S. Pat. No. 4,728,578, U.K. Patent Application G.B. 2193095 A, U.S. Pat. No. 4,728,575, U.S. Pat. No. 4,737,323, International Application PCT/US85/01161, Mayer et al., Biochimica et Biophysica Acta, Vol. 858, pp. 161-168 (1986), Hope et al., Biochimica et Biophysica Acta, Vol. 812, pp. 55-65 (1985), U.S. Pat. No. 4,533,254, Mahew et al., Methods In Enzymology, Vol. 149, pp. 64-77 (1987), Mahew et al., Biochimica et Biophysica Acta, Vol. 75, pp. 169-174 (1984), and Cheng et al., Investigative Radiology, Vol. 22, pp. 47-55 (1987), and U.S. Ser. No. 428,339, filed Oct. 27, 1989. The disclosures of each of the foregoing patents, publications and patent applications are incorporated by reference herein, in their entirety. A solvent free system similar to that described in International Application PCT/US85/01161, or U.S. Ser. No. 428,339, filed Oct. 27, 1989, may be employed in preparing the liposome constructions. By following these procedures, one is able to prepare liposomes having encapsulated therein a gaseous precursor or a solid or liquid contrast enhancing agent.

The materials which may be utilized in preparing the liposomes of the present invention include any of the materials or combinations thereof known to those skilled in the art as suitable in liposome construction. The lipids used may be of either natural or synthetic origin. Such materials include, but are not limited to, lipids with head groups including phosphatidylcholine, phosphatidylethanolamine, phosphatidylserine, phosphatidylglycerol, phosphatidic acid, phosphatidylinositol. Other lipids include lysolipids, fatty acids, sphingomyelin, glycosphingolipids, glucolipids, glycolipids, sulphatides, lipids with amide, ether, and ester-linked fatty acids, polymerizable lipids, and combinations thereof. Additionally, liposomes may include lipophilic compounds, such as cholesterol. As one skilled in the art will recognize, the liposomes may be synthesized in the absence or presence of incorporated glycolipid, complex carbohydrate, protein or synthetic polymer, using conventional procedures. The surface of a liposome may also be modified with a polymer, such as, for example, with polyethylene glycol (PEG), using procedures readily apparent to those skilled in the art. Lipids may contain functional surface groups for attachment to a metal, which provides for the chelation of radioactive isotopes or other materials that serve as the therapeutic entity. Any species of lipid may be used, with the sole proviso that the lipid or combination of lipids and associated materials incorporated within the lipid matrix should form a bilayer phase under physiologically relevant conditions. As one skilled in the art will recognize, the composition of the liposomes may be altered to modulate the biodistribution and clearance properties of the resulting liposomes.

The membrane bilayers in these structures typically encapsulate an aqueous volume, and form a permeability barrier between the encapsulated volume and the exterior solution. Lipids dispersed in aqueous solution spontaneously form bilayers with the hydrocarbon tails directed inward and the polar headgroups outward to interact with water. Simple agitation of the mixture usually produces multilamellar vesicles (MLVs), structures with many bilayers in an onion-like form having diameters of 1-10 μm (1000-10,000 nm). Sonication of these structures, or other methods known in the art, leads to formation of unilamellar vesicles (UVs) having an average diameter of about 30-300 nm. However, the range of 50 to 200 nm is considered to be optimal from the standpoint of, e.g., maximal circulation time in vivo. The actual equilibrium diameter is largely determined by the nature of the phospholipid used and the extent of incorporation of other lipids such as cholesterol. Standard methods for the formation of liposomes are known in the art, for example, methods for the commercial production of liposomes are described in U.S. Pat. No. 4,753,788 to Ronald C. Gamble and U.S. Pat. No. 4,935,171 to Kevin R. Bracken.

Either as MLVs or UVs, liposomes have proven valuable as vehicles for drug delivery in animals and in humans. Active drugs, including small hydrophilic molecules and polypeptides, can be trapped in the aqueous core of the liposome, while hydrophobic substances can be dissolved in the liposome membrane. Radioisotopes may be attached to the surfaces of vesicles and isotope-chelator complexes may be encapsulated in the interior of the vesicles. Other molecules, such as DNA or RNA, may be attached to the outside of the liposome for gene therapy applications. The liposome structure can be readily injected and form the basis for both sustained release and drug delivery to specific cell types, or parts of the body. MLVs, primarily because they are relatively large, are usually rapidly taken up by the reticuloendothelial system (the liver and spleen). The invention typically utilizes vesicles which remain in the circulatory system for hours and break down after internalization by the target cell. For these requirements the formulations preferably utilize UVs having a diameter of less than 200 nm, preferably less than 100 nm.

Linking Carriers

The term “linking carrier” refers to any entity which A) serves to link the therapeutic entity and the targeting entity, and B) confers additional advantageous properties to the vascular-targeted therapeutic agents other than merely keeping the therapeutic entity and the targeting entity in close proximity. Examples of these additional advantages include, but are not limited to: 1) multivalency, which is defined as the ability to attach either i) multiple therapeutic entities to the targeted therapeutic agents (i.e., several units of the same therapeutic entity, or one or more units of different therapeutic entities), which increases the effective “payload” of the therapeutic entity delivered to the targeted site; ii) multiple targeting entities to the targeted therapeutic agents (i.e., one or more units of different therapeutic entities, or, preferably, several units of the same targeting entity); or iii) both items i) and ii) of this sentence; and 2) improved circulation lifetimes, which can include tuning the size of the particle to achieve a specific rate of clearance by the reticuloendothelial system. The effective payload of therapeutic entity is the number of therapeutic entities delivered to the target site per binding event of the agent to the target. The payload will depend on the particular therapeutic entity and target. In some cases the payload will be as little as about 1 molecule delivered per binding event of the agent. In the case of a metal ion, the payload can be about one to 10³ molecules delivered per binding event. It is contemplated that the payload can be as high as 10⁴ molecules delivered per binding event. The payload can vary between about 1 to about 10⁴ molecules per binding event.

Preferred linking carriers are biocompatible polymers (such as dextran) or macromolecular assemblies of biocompatible components (such as liposomes). Examples of linking carriers include, but are not limited to, liposomes, polymerized liposomes, other lipid vesicles, dendrimers, polyethylene glycol assemblies, capped polylysines, poly(hydroxybutyric acid), dextrans, and coated polymers. A preferred linking carrier is a polymerized liposome. Polymerized liposomes are described in U.S. Pat. No. 5,512,294. Another preferred linking carrier is a dendrimer.

The linking carrier can be coupled to the targeting entity and the therapeutic entity by a variety of methods, depending on the specific chemistry involved. The coupling can be covalent or non-covalent. A variety of methods suitable for coupling of the targeting entity and the therapeutic entity to the linking carrier can be found in Hermanson, “Bioconjugate Techniques”, Academic Press: New York, 1996; and in “Chemistry of Protein Conjugation and Cross-linking” by S. S. Wong, CRC Press, 1993. Specific coupling methods include, but are not limited to, the use of bifunctional linkers, carbodiimide condensation, disulfide bond formation, and use of a specific binding pair where one member of the pair is on the linking carrier and another member of the pair is on the therapeutic or targeting entity, e.g. a biotin-avidin interaction.

Polymerized liposomes are self-assembled aggregates of lipid molecules which offer great versatility in particle size and surface chemistry. Polymerized liposomes are described in U.S. Pat. Nos. 5,512,294 and 6,132,764, incorporated by reference herein in their entirety. The hydrophobic tail groups of polymerizable lipids are derivatized with polymerizable groups, such as diacetylene groups, which irreversibly cross-link, or polymerize, when exposed to ultraviolet light or other radical, anionic or cationic, initiating species, while maintaining the distribution of functional groups at the surface of the liposome. The resulting polymerized liposome particle is stabilized against fusion with cell membranes or other liposomes and stabilized towards enzymatic degradation. The size of the polymerized liposomes can be controlled by extrusion or other methods known to those skilled in the art. Polymerized liposomes may be comprised of polymerizable lipids, but may also comprise saturated and non-alkyne, unsaturated lipids. The polymerized liposomes can be a mixture of lipids which provide different functional groups on the hydrophilic exposed surface. For example, some hydrophilic head groups can have functional surface groups, for example, biotin, amines, cyano, carboxylic acids, isothiocyanates, thiols, disulfides, a-halocarbonyl compounds, α,β-unsaturated carbonyl compounds and alkyl hydrazines. These groups can be used for attachment of targeting entities, such as antibodies, ligands, proteins, peptides, carbohydrates, vitamins, nucleic acids or combinations thereof for specific targeting and attachment to desired cell surface molecules, and for attachment of therapeutic entities, such as drugs, nucleic acids encoding genes with therapeutic effect or radioactive isotopes. Other head groups may have an attached or encapsulated therapeutic entity, such as, for example, antibodies, hormones and drugs for interaction with a biological site at or near the specific biological molecule to which the polymerized liposome particle attaches. Other hydrophilic head groups can have a functional surface group of diethylenetriamine pentaacetic acid, ethylenedinitrile tetraacetic acid, tetraazocyclododecane-1,4,7,10-tetraacetic acid (DOTA), porphoryin chelate and cyclohexane-1,2,-diamino-N,N′-diacetate, as well as derivatives of these compounds, for attachment to a metal, which provides for the chelation of radioactive isotopes or other materials that serve as the therapeutic entity. Examples of lipids with chelating head groups are provided in U.S. Pat. No. 5,512,294, incorporated by reference herein in its entirety.

Large numbers of therapeutic entities may be attached to one polymerized liposome that may also bear from several to about one thousand targeting entities for in vivo adherence to targeted surfaces. The improved binding conveyed by multiple targeting entities can also be utilized therapeutically to block cell adhesion to endothelial receptors in vivo. Blocking these receptors can be useful to control pathological processes, such as inflammation and control of metastatic cancer. For example, multi-valent sialyl Lewis X derivatized liposomes can be used to block neutrophil binding, and antibodies against VCAM-1 on polymerized liposomes can be used to block lymphocyte binding, e.g. T-cells.

The polymerized liposome particle can also contain groups to control nonspecific adhesion and reticuloendothelial system uptake. For example, PEGylation of liposomes has been shown to prolong circulation lifetimes; see International Patent Application WO 90/04384.

The component lipids of polymerized liposomes can be purified and characterized individually using standard, known techniques and then combined in controlled fashion to produce the final particle. The polymerized liposomes can be constructed to mimic native cell membranes or present functionality, such as ethylene glycol derivatives, that can reduce their potential immunogenicity. Additionally, the polymerized liposomes have a well-defined bilayer structure that can be characterized by known physical techniques such as transmission electron microscopy and atomic force microscopy.

Stabilizing Entities

The agents of the present invention preferably contain a stabilizing entity. As used herein, “stabilizing” refers to the ability to imparts additional advantages to the therapeutic or imaging agent, for example, physical stability, i.e., longer half-life, colloidal stability, and/or capacity for multivalency; that is, increased payload capacity due to numerous sites for attachment of targeting agents. As used herein, “stabilizing entity” refers to a macromolecule or polymer, which may optionally contain chemical functionality for the association of the stabilizing entity to the surface of the vesicle, and/or for subsequent association of therapeutic entities or targeting agents. The polymer should be biocompatible with aqueous solutions. Polymers useful to stabilize the liposomes of the present invention may be of natural, semi-synthetic (modified natural) or synthetic origin. A number of stabilizing entities which may be employed in the present invention are available, including xanthan gum, acacia, agar, agarose, alginic acid, alginate, sodium alginate, carrageenan, gelatin, guar gum, tragacanth, locust bean, bassorin, karaya, gum arabic, pectin, casein, bentonite, unpurified bentonite, purified bentonite, bentonite magma, and colloidal bentonite.

Other natural polymers include naturally occurring polysaccharides, such as, for example, arabinans, fructans, fucans, galactans, galacturonans, glucans, mannans, xylans (such as, for example, inulin), levan, fucoidan, carrageenan, galatocarolose, pectic acid, pectins, including amylose, pullulan, glycogen, amylopectin, cellulose, dextran, dextrose, dextrin, glucose, polyglucose, polydextrose, pustulan, chitin, agarose, keratin, chondroitin, dermatan, hyaluronic acid, alginic acid, xanthin gum, starch and various other natural homopolyner or heteropolymers, such as those containing one or more of the following aldoses, ketoses, acids or amines: erythrose, threose, ribose, arabinose, xylose, lyxose, allose, altrose, glucose, dextrose, mannose, gulose, idose, galactose, talose, erythrulose, ribulose, xylulose, psicose, fructose, sorbose, tagatose, mannitol, sorbitol, lactose, sucrose, trehalose, maltose, cellobiose, glycine, serine, threonine, cysteine, tyrosine, asparagine, glutamine, aspartic acid, glutamic acid, lysine, arginine, histidine, glucuronic acid, gluconic acid, glucaric acid, galacturonic acid, mannuronic acid, glucosamine, galactosamine, and neuraminic acid, and naturally occurring derivatives thereof. Other suitable polymers include proteins, such as albumin, polyalginates, and polylactide-glycolide copolymers, cellulose, cellulose (microcrystalline), methylcellulose, hydroxyethylcellulose, hydroxypropylcellulose, hydroxypropylmethylcellulose, carboxymethylcellulose, and calcium carboxymethylcellulose.

Exemplary semi-synthetic polymers include carboxymethylcellulose, sodium carboxymethylcellulose, carboxymethylcellulose sodium 12, hydroxymethylcellulose, hydroxypropylmethylcellulose, methylcellulose, and methoxycellulose. Other semi-synthetic polymers suitable for use in the present invention include carboxydextran, aminodextran, dextran aldehyde, chitosan, and carboxymethyl chitosan.

Exemplary synthetic polymers include poly(ethylene imine) and derivatives, polyphosphazenes, hydroxyapatites, fluoroapatite polymers, polyethylenes (such as, for example, polyethylene glycol, the class of compounds referred to as Pluronics®, commercially available from BASF, (Parsippany, N.J.), polyoxyethylene, and polyethylene terephthlate), polypropylenes (such as, for example, polypropylene glycol), polyurethanes (such as, for example, polyvinyl alcohol (PVA), polyvinyl chloride and polyvinylpyrrolidone), polyamides including nylon, polystyrene, polylactic acids, fluorinated hydrocarbon polymers, fluorinated carbon polymers (such as, for example, polytetrafluoroethylene), acrylate, methacrylate, and polymethylmethacrylate, and derivatives thereof, polysorbate, carbomer 934P, magnesium aluminum silicate, aluminum monostearate, polyethylene oxide, polyvinylalcohol, povidone, polyethylene glycol, and propylene glycol. Methods for the preparation of vesicles which employ polymers to stabilize vesicle compositions will be readily apparent to one skilled in the art, in view of the present disclosure, when coupled with information known in the art, such as that described and referred to in Unger, U.S. Pat. No. 5,205,290, the disclosure of which is hereby incorporated by reference herein in its entirety.

In a preferred embodiment, the stabilizing entity is dextran. In another preferred embodiment, the stabilizing entity is a modified dextran, such as amino dextran. In a further preferred embodiment, the stabilizing entity is poly(ethylene imine) (PEI). Without being bound by theory, it is believed that dextran may increase circulation times of liposomes in a manner similar to PEG. Additionally, each polymer chain (i.e. aminodextran or succinylated aminodextran) contains numerous sites for attachment of targeting agents, providing the ability to increase the payload of the entire lipid construct. This ability to increase the payload differentiates the stabilizing agents of the present invention from PEG. For PEG there is only one site of attachment, thus the targeting agent loading capacity for PEG (with a single site for attachment per chain) is limited relative to a polymer system with multiple sites for attachment.

In other preferred embodiments, the following polymers and their derivatives are used. poly(galacturonic acid), poly(L-glutamic acid), poly(L-glutamic acid-L-tyrosine), poly[R)-3-hydroxybutyric acid], poly(inosinic acid potassium salt), poly(L-lysine), poly(acrylic acid), poly(ethanolsulfonic acid sodium salt), poly(methylhydrosiloxane), poly(vinyl alcohol), poly(vinylpolypyrrolidone), poly(vinylpyrrolidone), poly(glycolide), poly(lactide), poly(lactide-co-glycolide), and hyaluronic acid. In other preferred embodiments, copolymers including a monomer having at least one reactive site, and preferably multiple reactive sites, for the attachment of the copolymer to the vesicle or other molecule.

In some embodiments, the polymer may act as a hetero- or homobifunctional linking agent for the attachment of targeting agents, therapeutic entities, proteins or chelators such as DTPA and its derivatives.

In one embodiment, the stabilizing entity is associated with the vesicle by covalent means. In another embodiment, the stabilizing entity is associated with the vesicle by non-covalent means. Covalent means for attaching the targeting entity with the liposome are known in the art and described in the EXAMPLES section.

Noncovalent means for attaching the targeting entity with the liposome include but are not limited to attachment via ionic, hydrogen-bonding interactions, including those mediated by water molecules or other solvents, hyrdophobic interactions, or any combination of these.

In a preferred embodiment, the stabilizing agent forms a coating on the liposome.

Targeting Entities

The term “targeting entity” refers to a molecule, macromolecule, or molecular assembly which binds specifically to a biological target. Examples of targeting entities include, but are not limited to, antibodies (including antibody fragments and other antibody-derived molecules which retain specific binding, such as Fab, F(ab′)2, Fv, and scFv derived from antibodies); receptor-binding ligands, such as hormones or other molecules that bind specifically to a receptor; cytokines, which are polypeptides that affect cell function and modulate interactions between cells associated with immune, inflammatory or hematopoietic responses; molecules that bind to enzymes, such as enzyme inhibitors; nucleic acid ligands or aptamers, and one or more members of a specific binding interaction such as biotin or iminobiotin and avidin or streptavidin. Preferred targeting entities are molecules which specifically bind to receptors or antigens found on vascular cells. More preferred are molecules which specifically bind to receptors, antigens or markers found on cells of angiogenic neovasculature or receptors, antigens or markers associated with tumor vasculature. The receptors, antigens or markers associated with tumor vasculature can be expressed on cells of vessels which penetrate or are located within the tumor, or which are confined to the inner or outer periphery of the tumor. In one embodiment, the invention takes advantage of pre-existing or induced leakage from the tumor vascular bed; in this embodiment, tumor cell antigens can also be directly targeted with agents that pass from the circulation into the tumor interstitial volume.

Other targeting entities target endothelial receptors, tissue or other targets accessible through a body fluid or receptors or other targets upregulated in a tissue or cell adjacent to or in a bodily fluid. For example, stabilizing entities attached to carriers designed to deliver drugs to the eye can be injected into the vitreous, choroid, or sclera; or targeting agents attached to carriers designed to deliver drugs to the joint can be injected into the synovial fluid.

Targeting entities attached to the polymerized liposomes, or linking carriers of the invention include, but are not limited to, small molecule ligands, such as carbohydrates, and compounds such as those disclosed in U.S. Pat. No. 5,792,783 (small molecule ligands are defined herein as organic molecules with a molecular weight of about 1000 daltons or less, which serve as ligands for a vascular target or vascular cell marker); proteins, such as antibodies and growth factors; peptides, such as RGD-containing peptides (e.g. those described in U.S. Pat. No. 5,866,540), bombesin or gastrin-releasing peptide, peptides selected by phage-display techniques such as those described in U.S. Pat. No. 5,403,484, and peptides designed de novo to be complementary to tumor-expressed receptors; antigenic determinants; or other receptor targeting groups. These head groups can be used to control the biodistribution, non-specific adhesion, and blood pool half-life of the polymerized liposomes. For example, β-D-lactose has been attached on the surface to target the asialoglycoprotein (ASG) found in liver cells which are in contact with the circulating blood pool. Glycolipids can be derivatized for use as targeting entities by converting the commercially available lipid (DAGPE) or the PEG-PDA amine into its isocyanate followed by treatment with triethylene glycol diamine spacer to produce the amine terminated thiocarbamate lipid which by treatment with the para-isothiocyanophenyl glycoside of the carbohydrate ligand produces the desired targeting glycolipids. This synthesis provides a water-soluble flexible spacer molecule spaced between the lipid that will form the internal structure or core of the liposome and the ligand that binds to cell surface receptors, allowing the ligand to be readily accessible to the protein receptors on the cell surfaces. The carbohydrate ligands can be derived from reducing sugars or glycosides, such as para-nitrophenyl glycosides, a wide range of which are commercially available or easily constructed using chemical or enzymatic methods. Polymerized liposomes coated with carbohydrate ligands can be produced by mixing appropriate amounts of individual lipids followed by sonication, extrusion and polymerization and filtration as described above. Suitable carbohydrate derivatized polymerized liposomes have about 1 to about 30 mole percent of the targeting glycolipid and filler lipid, such as PDA, DAPC or DAPE, with the balance being metal chelated lipid. Other lipids may be included in the polymerized liposomes to assure liposome formation and provide high contrast and recirculation.

In some embodiments, the targeting entity targets the liposomes to a cell surface. Delivery of the therapeutic or imaging agent can occur through endocytosis of the liposomes. Such deliveries are known in the art. See, for example, Mastrobattista, et al., Immunoliposomes for the Targeted Delivery of Antitumor Drugs, Adv. Drug Del. Rev. (1999) 40:103-27.

In a preferred embodiment, the targeting entity is attached to the stabilizing entity. In one embodiment, the attachment is by covalent means. In another embodiment, the attachment is by non-covalent means. For example, antibody targeting entities may be attached by a biotin-avidin biotinylated antibody sandwich, to allow a variety of commercially available biotinylated antibodies to be used on the coated polymerized liposome. Specific vasculature targeting agents of use in the invention include (but are not limited to) anti-VCAM-1 antibodies (VCAM=vascular cell adhesion molecule); anti-ICAM-1 antibodies (ICAM=intercellular adhesion molecule); anti-integrin antibodies (e.g., antibodies directed against αᵥβ₃ integrins such as LM609, described in International Patent Application WO 89/05155 and Cheresh et al. J. Biol. Chem. 262:17703-11 (1987), and Vitaxin, described in International Patent Application WO 9833919 and in Wu et al., Proc. Natl. Acad. Sci. U.S. Pat. No. 95(11):6037-42 (1998); and antibodies directed against P- and E-selectins, pleiotropin and endosialin, endoglin, VEGF receptors, PDGF receptors, EGF receptors, FGF receptors, MMPs, and prostate specific membrane antigen (PSMA). Additional targets are described by E. Ruoslahti in Nature Reviews: Cancer, 2, 83-90 (2002).

In one embodiment of the invention, the vascular-targeted therapeutic agent is combined with an agent targeted directly towards tumor cells. This embodiment takes advantage of the fact that the neovasculature surrounding tumors is often highly permeable or “leaky,” allowing direct passage of materials from the bloodstream into the interstitial space surrounding the tumor. Alternatively, the vascular-targeted therapeutic agent itself can induce permeability in the tumor vasculature. For example, when the agent carries a radioactive therapeutic entity, upon binding to the vascular tissue and irradiating that tissue, cell death of the vascular epithelium will follow and the integrity of the vasculature will be compromised.

Accordingly, in one embodiment, the vascular-targeted therapeutic agent has two targeting entities: a targeting entity directed towards a vascular marker, and a targeting entity directed towards a tumor cell marker. In another embodiment, an antitumor agent is administered with the vascular-targeted therapy agent. The antitumor agent can be administered simultaneously with the vascular-targeted therapy agent, or subsequent to administration of the vascular-targeted therapy agent. In particular, when the vascular-targeted therapy agent is relied upon to compromise vascular integrity in the area of the tumor, administration of the antitumor agent is preferably done at the point of maximum damage to the tumor vasculature.

The antitumor agent can be a conventional antitumor therapy, such as cisplatin; antibodies directed against tumor markers, such as anti-Her2/neu antibodies (e.g., Herceptin); or tripartite agents, such as those described herein for vascular-targeted therapeutic agents, but targeted against the tumor cell rather than the vasculature. A summary of monoclonal antibodies directed against various tumor markers is given in Table I of U.S. Pat. No. 6,093,399, hereby incorporated by reference herein in its entirety. In general, when the vascular-targeted therapy agent compromises vascular integrity in the area of the tumor, the effectiveness of any drug which operates directly on the tumor cells can be enhanced.

The size of the vesicles can be adjusted for the particular intended end use including, for example, diagnostic and/or therapeutic use. As the size of the linking carrier can be manipulated readily, the overall size of the vascular-targeted therapeutic agents can be adapted for optimum passage of the particles through the permeable (“leaky”) vasculature at the site of pathology, as long as the agent retains sufficient size to maintain its desired properties (e.g., circulation lifetime, multivalency). Accordingly, the particles can be sized at 30, 50, 100, 150, 200, 250, 300 or 350 nm in size, as desired. In addition, the size of the particles can be chosen so as to permit a first administration of particles of a size that cannot pass through the permeable vasculature, followed by one or more additional administrations of particles of a size that can pass through the permeable vasculature. The size of the vesicles may preferably range from about 30 nanometers (nm) to about 400 nm in diameter, and all combinations and subcombinations of ranges therein. More preferably, the vesicles have diameters of from about 10 nm to about 500 nm, with diameters from about 40 nm to about 120 nm being even more preferred. In connection with particular uses, for example, intravascular use, including magnetic resonance imaging of the vasculature, it may be preferred that the vesicles be no larger than about 500 nm in diameter, with smaller vesicles being preferred, for example, vesicles of no larger than about 100 nm in diameter. It is contemplated that these smaller vesicles may perfuse small vascular channels, such as the microvasculature, while at the same time providing enough space or room within the vascular channel to permit red blood cells to slide past the vesicles.

While one major focus of the invention is the use of vascular-targeted therapy agent against the vasculature of tumors in order to treat cancer, the agents of the invention can be used in any disease where neovascularization or other aberrant vascular growth accompanies or contributes to pathology. Diseases associated with neovascular growth include, but are not limited to, solid tumors; blood born tumors such as leukemias; tumor metastasis; benign tumors, for example hemangiomas, acoustic neuromas, neurofibromas, trachomas, and pyogenic granulomas; rheumatoid arthritis; psoriasis; chronic inflammation; ocular angiogenic diseases, for example, diabetic retinopathy, retinopathy of prematurity, macular degeneration, corneal graft rejection, neovascular glaucoma, retrolental fibroplasia, rubeosis; arteriovenous malformations; ischemic limb angiogenesis; Osler-Webber Syndrome; myocardial angiogenesis; plaque neovascularization; telangiectasia; hemophiliac joints; angiofibroma; and wound granulation. Diseases of excessive or abnormal stimulation of endothelial cells include, but are not limited to, intestinal adhesions, atherosclerosis, restenosis, scleroderma, and hypertrophic scars, i.e., keloids.

Differing administration vehicles, dosages, and routes of administration can be determined for optimal administration of the agents; for example, injection near the site of a tumor may be preferable for treating solid tumors. Therapy of these disease states can also take advantage of the permeability of the neovasulature at the site of the pathology, as discussed above, in order to specifically deliver the vascular-targeted therapeutic agents to the interstitial space at the site of pathology.

Therapeutic Compositions

The present invention is also directed toward therapeutic compositions comprising the therapeutic agents of the present invention. Compositions of the present invention can also include other components such as a pharmaceutically acceptable excipient, an adjuvant, and/or a carrier. For example, compositions of the present invention can be formulated in an excipient that the animal to be treated can tolerate. Examples of such excipients include water, saline, Ringer's solution, dextrose solution, mannitol, Hank's solution, and other aqueous physiologically balanced salt solutions. Nonaqueous vehicles, such as fixed oils, sesame oil, ethyl oleate, or triglycerides may also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer, Tris buffer, histidine, citrate, and glycine, or mixtures thereof, while examples of preservatives include thimerosal, m- or o-cresol, formalin and benzyl alcohol. Standard formulations can either be liquid injectables or solids which can be taken up in a suitable liquid as a suspension or solution for injection. Thus, in a non-liquid formulation, the excipient can comprise dextrose, human serum albumin, preservatives, etc., to which sterile water or saline can be added prior to administration.

In one embodiment of the present invention, the composition can also include an immunopotentiator, such as an adjuvant or a carrier. Adjuvants are typically substances that generally enhance the immune response of an animal to a specific antigen. Suitable adjuvants include, but are not limited to, Freund's adjuvant; other bacterial cell wall components; aluminum-based salts; calcium-based salts; silica; polynucleotides; toxoids; serum proteins; viral coat proteins; other bacterial-derived preparations; gamma interferon; block copolymer adjuvants, such as Hunter's Titermax adjuvant (Vaxcel.TM., Inc. Norcross, Ga.); Ribi adjuvants (available from Ribi ImmunoChem Research, Inc., Hamilton, Mont.); and saponins and their derivatives, such as Quil A (available from Superfos Biosector A/S, Denmark). Carriers are typically compounds that increase the half-life of a therapeutic composition in the treated animal. Suitable carriers include, but are not limited to, polymeric controlled release formulations, biodegradable implants, liposomes, bacteria, viruses, oils, esters, and glycols.

One embodiment of the present invention is a controlled release formulation that is capable of slowly releasing a composition of the present invention into an animal. As used herein, a controlled release formulation comprises a composition of the present invention in a controlled release vehicle. Suitable controlled release vehicles include, but are not limited to, biocompatible polymers, other polymeric matrices, capsules, microcapsules, microparticles, bolus preparations, osmotic pumps, diffusion devices, liposomes, lipospheres, and transdermal delivery systems. Other controlled release formulations of the present invention include liquids that, upon administration to an animal, form a solid or a gel in situ. Preferred controlled release formulations are biodegradable (i.e., bioerodible).

Generally, the therapeutic agents used in the invention are administered to an animal in an effective amount. Generally, an effective amount is an amount effective to either (1) reduce the symptoms of the disease sought to be treated or (2) induce a pharmacological change relevant to treating the disease sought to be treated. For cancer, an effective amount includes an amount effective to: reduce the size of a tumor; slow the growth of a tumor; prevent or inhibit metastases; or increase the life expectancy of the affected animal.

Therapeutically effective amounts of the therapeutic agents can be any amount or doses sufficient to bring about the desired effect and depend, in part, on the condition, type and location of the cancer, the size and condition of the patient, as well as other factors readily known to those skilled in the art. The dosages can be given as a single dose, or as several doses, for example, divided over the course of several weeks.

The present invention is also directed toward methods of treatment utilizing the therapeutic compositions of the present invention. The method comprises administering the therapeutic agent to a subject in need of such administration.

The therapeutic agents of the instant invention can be administered by any suitable means, including, for example, parenteral, topical, oral or local administration, such as intradermally, by injection, or by aerosol. In the preferred embodiment of the invention, the agent is administered by injection. Such injection can be locally administered to any affected area. A therapeutic composition can be administered in a variety of unit dosage forms depending upon the method of administration. For example, unit dosage forms suitable for oral administration of an animal include powder, tablets, pills and capsules. Preferred delivery methods for a therapeutic composition of the present invention include intravenous administration and local administration by, for example, injection or topical administration. For particular modes of delivery, a therapeutic composition of the present invention can be formulated in an excipient of the present invention. A therapeutic reagent of the present invention can be administered to any animal, preferably to mammals, and more preferably to humans.

The particular mode of administration will depend on the condition to be treated. It is contemplated that administration of the agents of the present invention may be via any bodily fluid, or any target or any tissue accessible through a body fluid.

Preferred routes of administration of the cell-surface targeted therapeutic agents of the present invention are by intravenous, interperitoneal, or subcutaneous injection including administration to veins or the lymphatic system. While the primary focus of the invention is on vascular-targeted agents, in principle, a targeted agent can be designed to focus on markers present in other fluids, body tissues, and body cavities, e.g. synovial fluid, ocular fluid, or spinal fluid. Thus, for example, an agent can be administered to spinal fluid, where an antibody targets a site of pathology accessible from the spinal fluid. Intrathecal delivery, that is, administration into the cerebrospinal fluid bathing the spinal cord and brain, may be appropriate for example, in the case of a target residing in the choroid plexus endothelium of the cerebral spinal fluid (CSF)-blood barrier.

As an example of one treatment route of administration through a bodily fluid is one in which the disease to be treated is rheumatoid arthritis. In this embodiment of the invention, the invention provides therapeutic agents to treat inflamed synovia of people afflicted with rheumatoid arthritis. This type of therapeutic agent is a radiation synovectomy agent. Individuals with rheumatoid arthritis experience destruction of the diarthroidal or synovial joints, which causes substantial pain and physical disability. The disease will involve the hands (metacarpophalangeal joints), elbows, wrists, ankles and shoulders for most of these patients, and over half will have affected knee joints. Untreated, the joint linings become increasingly inflamed resulting in pain, loss of motion and destruction of articular cartilage. Chemicals, surgery, and radiation have been used to attack and destroy or remove the inflamed synovium, all with drawbacks.

The concentration of the radiation synovectomy agent varies with the particular use, but a sufficient amount is present to provide satisfactory radiation synovectomy. For example, in radiation synovectomy of the hip, the concentration of the agent will generally be higher than when used for the radiation synovectomy of the wrist joints. The radiation synovectomy composition is administered so that preferably it remains substantially in the joint for 20 half-lifes of the isotope although shorter residence times are acceptable as long as the leakage of the radionuclide is small and the leaked radionuclide is rapidly cleared from the body.

The radiation synovectomy compositions may be used in the usual way for such procedures. For example, in the case of the treatment of a knee-joint, a sufficient amount of the radiation synovectomy composition to provide adequate radiation synovectomy is injected into the knee-joint. There are a number of different techniques which can be used and the appropriate technique varies on the joint being treated. An example for the knee joint can be found, for example, in Nuclear Medicine Therapy, J. C. Harbert, J. S. Robertson and K. D. Reid, 1987, Thieme Medical Publishers, pages 172-3.

The route of administration through the synovia may also be useful in the treatment of osteoarthritis. Osteoarthritis is a disease where cartilage degradation leads to severe pain and inability to use the affected joint. Although age is the single most powerful risk factor, major trauma and repetitive joint use are additional risk factors. Major features of the disease include thinning of the joint, softening of the cartilage, cartilage ulcers, and abraded bone. Delivery of agents by injection of targeted carriers to synovial fluid to reduce inflammation, inhibit degradative enzymes, and decrease pain are envisioned in this embodiment of the invention.

Another route of administration is through ocular fluid. In the eye, the retina is a thin layer of light-sensitive tissue that lines the inside wall of the back of the eye. When light enters the eye, it is focused by the cornea and the lens onto the retina. The retina then transforms the light images into electrical impulses that are sent to the brain through the optic nerve.

The macula is a very small area of the retina responsible for central vision and color vision. The macula allows us to read, drive, and perform detailed work. Surrounding the macula is the peripheral retina which is responsible for side vision and night vision. Macular degeneration is damage or breakdown of the macula, underlying tissue, or adjacent tissue. Macular degeneration is the leading cause of decreased visual acuity and impairment of reading and fine “close-up” vision. Age-related macular degeneration (ARMD) is the most common cause of legal blindness in the elderly.

The most common form of macular degeneration is called “dry” or involutional macular degeneration and results from the thinning of vascular and other structural or nutritional tissues underlying the retina in the macular region. A more severe form is termed “wet” or exudative macular degeneration. In this form, blood vessels in the choroidal layer (a layer underneath the retina and providing nourishment to the retina) break through a thin protective layer between the two tissues. These blood vessels may grow abnormally directly beneath the retina in a rapid uncontrolled fashion, resulting in oozing, bleeding, or eventually scar tissue formation in the macula which leads to severe loss of central vision. This process is termed choroidal neovascularization (CNV).

CNV is a condition that has a poor prognosis; effective treatment using thermal laser photocoagulation relies upon lesion detection and resultant mapping of the borders. Angiography is used to detect leakage from the offending vessels but often CNV is larger than indicated by conventional angiograms since the vessels are large, have an ill-defined bed, protrude below into the retina and can associate with pigmented epithelium.

Neovascularization results in visual loss in other eye diseases including neovascular glaucoma, ocular histoplasmosis syndrome, myopia, diabetes, pterygium, and infectious and inflammatory diseases. In histoplasmosis syndrome, a series of events occur in the choroidal layer of the inside lining of the back of the eye resulting in localized inflammation of the choroid and consequent scarring with loss of function of the involved retina and production of a blind spot (scotoma). In some cases, the choroid layer is provoked to produce new blood vessels that are much more fragile than normal blood vessels. They have a tendency to bleed with additional scarring, and loss of function of the overlying retina. Diabetic retinopathy involves retinal rather than choroidal blood vessels resulting in hemorrhages, vascular irregularities, and whitish exudates. Retinal neovascularization may occur in the most severe forms. When the vasculature of the eye is targeted, it should be appreciated that targets may be present on either side of the vasculature.

Delivery of the agents of the present invention to the tissues of the eye can be in many forms, including intravenous, ophthalmic, and topical. For ophthalmic topical administration, the agents of the present invention can be prepared in the form of aqueous eye drops such as aqueous suspended eye drops, viscous eye drops, gel, aqueous solution, emulsion, ointment, and the like. Additives suitable for the preparation of such formulations are known to those skilled in the art. In the case of a sustained-release delivery system for the eye, the sustained-release delivery system may be placed under the eyelid or injected into the conjunctiva, sclera, retina, optic nerve sheath, or in an intraocular or intraorbitol location. Intravitreal delivery of agents to the eye is also contemplated. Such intravitreal delivery methods are known to those of skill in the art. The delivery may include delivery via a device, such as that described in U.S. Pat. No. 6,251,090 to Avery.

In a further embodiment, the therapeutic agents of the present invention are useful for gene therapy. As used herein, the phrase “gene therapy” refers to the transfer of genetic material (e.g., DNA or RNA) of interest into a host to treat or prevent a genetic or acquired disease or condition. The genetic material of interest encodes a product (e.g., a protein polypeptide, peptide or functional RNA) whose production in vivo is desired. For example, the genetic material of interest can encode a hormone, receptor, enzyme or polypeptide of therapeutic value. In a specific embodiment, the subject invention utilizes a class of lipid molecules for use in non-viral gene therapy which can complex with nucleic acids as described in Hughes, et al., U.S. Pat. No. 6,169,078, incorporated by reference herein in its entirety, in which a disulfide linker is provided between a polar head group and a lipophilic tail group of a lipid.

These therapeutic compounds of the present invention effectively complex with DNA and facilitate the transfer of DNA through a cell membrane into the intracellular space of a cell to be transformed with heterologous DNA. Furthermore, these lipid molecules facilitate the release of heterologous DNA in the cell cytoplasm thereby increasing gene transfection during gene therapy in a human or animal.

Cationic lipid-polyanionic macromolecule aggregates may be formed by a variety of methods known in the art. Representative methods are disclosed by Felgner et al., supra; Eppstein et al. supra; Behr et al. supra; Bangham, A. et al. M. Mol. Biol. 23:238, 1965; Olson, F. et al. Biochim. Biophys. Acta 557:9, 1979; Szoka, F. et: al. Proc. Natl. Acad. Sci. 75: 4194, 1978; Mayhew, E. et al. Biochim. Biophys. Acta 775:169, 1984; Kim, S. et al. Biochim. Biophys. Acta 728:339, 1983; and Fukunaga, M. et al. Endocrinol. 115:757, 1984. In general aggregates may be formed by preparing lipid particles consisting of either (1) a cationic lipid or (2) a cationic lipid mixed with a colipid, followed by adding a polyanionic macromolecule to the lipid particles at about room temperature (about 18 to 26° C.). In general, conditions are chosen that are not conducive to deprotection of protected groups. In one embodiment, the mixture is then allowed to form an aggregate over a period of about 10 minutes to about 20 hours, with about 15 to 60 minutes most conveniently used. Other time periods may be appropriate for specific lipid types. The complexes may be formed over a longer period, but additional enhancement of transfection efficiency will not usually be gained by a longer period of complexing.

The compounds and methods of the subject invention can be used to intracellularly deliver a desired molecule, such as, for example, a polynucleotide, to a target cell. The desired polynucleotide can be composed of DNA or RNA or analogs thereof. The desired polynucleotides delivered using the present invention can be composed of nucleotide sequences that provide different functions or activities, such as nucleotides that have a regulatory function, e.g., promoter sequences, or that encode a polypeptide. The desired polynucleotide can also provide nucleotide sequences that are antisense to other nucleotide sequences in the cell. For example, the desired polynucleotide when transcribed in the cell can provide a polynucleotide that has a sequence that is antisense to other nucleotide sequences in the cell. The antisense sequences can hybridize to the sense strand sequences in the cell. Polynucleotides that provide antisense sequences can be readily prepared by the ordinarily skilled artisan. The desired polynucleotide delivered into the cell can also comprise a nucleotide sequence that is capable of forming a triplex complex with double-stranded DNA in the cell.

Imaging

The present invention is directed to imaging agents displaying important properties in medical diagnosis. More particularly, the present invention is directed to magnetic resonance imaging contrast agents, such as gadolinium, ultrasound imaging agents, or nuclear imaging agents, such as Tc-99m, In-111, Ga-67, Rh-105, I-123, Nd-147, Pm-151, Sm-153, Gd-159, Tb-161, Er-171, Re-186, Re-188, and T1-201.

This invention also provides a method of diagnosing abnormal pathology in vivo comprising, introducing a plurality of targeting image enhancing polymerized particles targeted to a molecule involved in the abnormal pathology into a bodily fluid contacting the abnormal pathology, the targeting image enhancing polymerized particles attaching to a molecule involved in the abnormal pathology, and imaging in vivo the targeting image enhancing polymerized particles attached to molecules involved in the abnormal pathology.

Diagnostics

The present invention further provides methods and reagents for diagnostic purposes. Diagnostic assays contemplated by the present invention include, but are not limited to, receptor-binding assays, antibody assays, immunohistochemical assays, flow cytometry assays, genomics and nucleic acid detection assays. High-throughput screening arrays and assays are also contemplated.

This invention provides various methods for in vitro assays. For example, antibody-conjugated polymerized liposomes, according to this invention, provide an ultra-sensitive diagnostic assay for specific antigens in solution. Polymerized liposomes of this invention having a chelator head group chelated to spectroscopically distinct ions provide high sensitivity for immunoassays as well as ligand and receptor-based assays. Polymerized liposomes of this invention having a fluorophore head group provide a method for detection of glycoproteins on cell surfaces.

Liposomes useful in diagnostic assays are described in U.S. Pat. No. 6,090,408, entitled “Use of Polymerized Lipid Diagnostic Agents,” and U.S. Pat. No. 6,132,764, entitled “Targeted Polymerized Liposome Diagnostic and Treatment Agents,” each incorporated by reference herein in its entirety.

In one embodiment of this invention, a targeting polymerized liposome particle comprises: an assembly of a plurality of liposome forming lipids each having an active hydrophilic head group linked by a bifunctional linker portion to the liposome forming lipid, and a hydrophobic tail group having a polymerizable functional group polymerized with a polymerizable functional group of an adjacent hydrophobic tail group of one of the plurality of liposome forming lipids, at least a portion of the hydrophilic head groups having an attached targeting active agent for attachment to a specific biological molecule. In another embodiment, the targeting polymerized liposome particle has a second portion of the hydrophilic head groups with functional surface groups attached to an image contrast enhancement agent to form a targeting image enhancing polymerized liposome particle. In yet another embodiment, a portion of the hydrophilic head groups have functional surface groups attached to or encapsulating a treatment agent for interaction with a biological site at or near the specific biological molecule to which the particle attaches, forming a targeting delivery polymerized liposome particle or a targeting image enhancing delivery polymerized liposome particle.

This invention provides a method of assaying abnormal pathology in vitro comprising, introducing a plurality of liposomes of the present invention to a molecule involved in the abnormal pathology into a fluid contacting the abnormal pathology, the targeting polymerized liposome particles attaching to a molecule involved in the abnormal pathology, and detecting in vitro the targeting polymerized liposome particles attached to molecules involved in the abnormal pathology.

Exemplary Lipid Constructs and Uses

Stabilized Vesicles

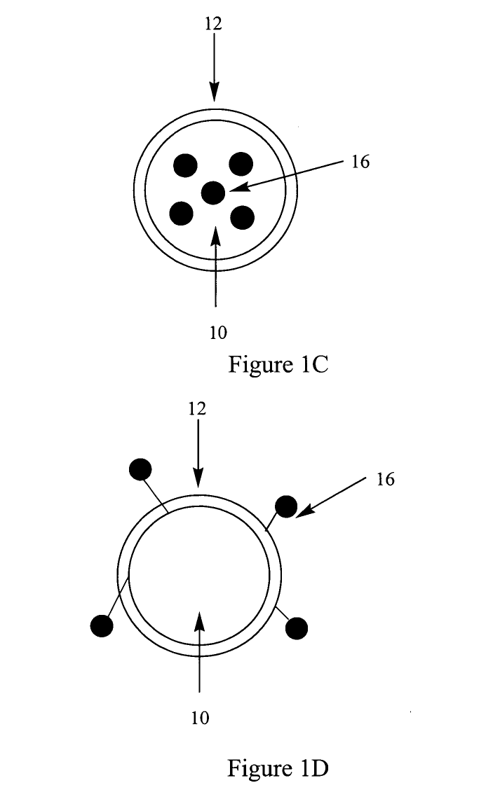

Vesicles prepared as described in Examples 1 and 2, contain diacetylene lipids 1,2-bis(10,12-tricosadiynoyl)-sn-glycero-3-phosphocholine (BisT-PC, 1) (FIG. 2) and diethylenetriaminetriacetic acid (DTTA) lipid derivative (2) (FIG. 2). Diacetylenic lipids may be cross-linked during exposure to UV light resulting in a highly conjugated backbone consisting of alternating double and triple carbon-carbon bonds (D. S. Johnston, S. Sanghera, M. Pons, D. Chapman, Biochim Biophys Acta 602, 57-69. (1980)). Dextran-based, and poly (ethylene imine) stabilizing agents were attached to the surface of the non-polymerized liposomes or the polymerized vesicles using EDAC chemistry as described in Examples 2 and 8.

Attachment of Antibodies to Vesicles

Antibodies including murine antibody LM609 (P. C. Brooks, et al., J Clin Invest 96, 1815-22 (1995)) or the humanized antibody Vitaxin (H. Wu, et al., Proc Natl Acad Sci USA 95, 6037-42 (1998)), each of which bind the human αᵥβ₃ integrin, are attached to the surface carboxyl groups of the polymerized vesicles using EDAC chemistry as described in Examples 2C, which results primarily in amide bond formation with nucleophilic groups such as the amines on N-terminus amino groups or lysines that are present on the protein or peptide (G. T. Hermanson, Bioconjugate Techniques (Academic Press, San Diego, 1996)).

Attachment of Metals to the Vesicles

Yttrium-90 is attached to the polymerized vesicles or liposomes via chelation to the triacetic acid DTTA or DPTA head group of the respective lipid derivatives as described in Examples 1 and 2. Previous studies have shown that the metal binding capacities of PVs and Vitaxin-PVs are indistinguishable, thus the use of EDAC does not significantly alter the concentration of chelating groups under the conditions used to attach antibodies and peptides.

In-vitro Targeting of Integrin-targeted Vesicles

Vitaxin-PV conjugates, which also bind yttrium-90 with high efficiency, target the αᵥβ₃ integrin in-vitro in a radiometric binding assay performed as described in Example 7. Previous studies have shown a linear response in signal as a function of vesicle concentration with signal to background ratios of up to 270 to 1. The present results indicate that dextran-coated vesicles provide an even higher delivery potential, up to eight-fold higher than unstabilized vesicles.

Stability of Stabilized Conjugates In-vitro

In order to assess the stability of conjugates in serum, the stabilized and unstabilized vesicle complexes were incubated in rabbit serum at 37° C. and compared. Previous studies have indicated that Vitaxin-PV conjugates are significantly more stable than corresponding unpolymerized liposomes, having a greater half-life and higher ⁹⁰Y signals. The present results indicate that dextran-coated vesicles provide more stabilization, retaining 5-6 times more ⁹⁰Y than unstabilized vesicles.

The present studies also indicate that the dextran-coated vesicles exhibit enhanced colloidal stability. That is, dextran-stabilized vesicles do not undergo a significant change in size in the presence of added salt, while the mean diameter of unstabilized vesicles inceases by three-fold in thirty minutes in the presence of added salt.

Treatment of Melanoma in a Murine Tumor Model

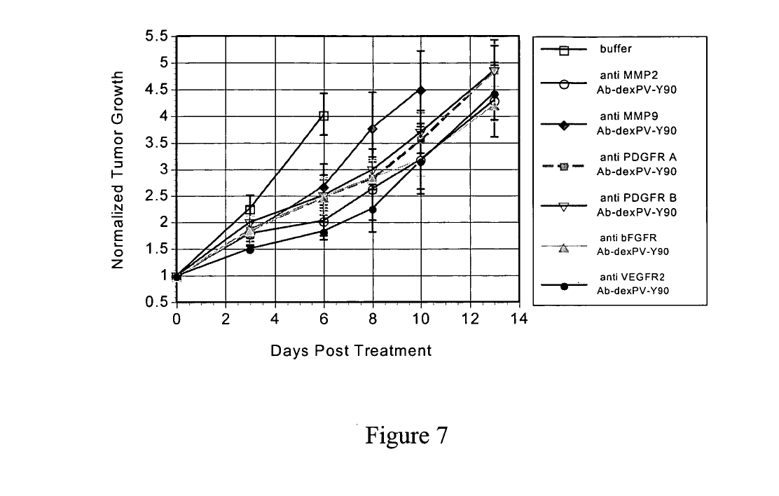

Example 10 describes the treatment of a melanoma murine tumor model with stabilized therapeutic agents of the present invention. FIG. 7 shows that the stabilized lipid constructs reduce tumor growth.

### EXAMPLES

### Example 1

### Procedure for the Preparation of Liposomes or Polymerized Vesicles

A. Procedure for the preparation of polymerized vesicles. Vesicles were prepared by extrusion or by homogenization using a Microfluidics homogenizer. To a 100 mL flask was added diethylenetriaminetriacetic acid (DTTA) lipid derivative 3 (15 mg) in chloroform (3 mL) and 1,2-bis(10,12-tricosadiynoyl)-sn-glycero-phosphocholine, BisT-PC 2 (220 mg) in chloroform (11 mL). Solvent was removed at ≈60° C. by rotary evaporation. Water (10 mL) was added and the solution was frozen on a dry ice/acetone mixture until solid. The solution was thawed at 60° C. and the pH was adjusted to 8 by adding 20 μL of 0.5 M NaOH. The freeze-thaw process was repeated until a translucent solution was obtained. This solution was passed through a 30 nm polycarbonate filter in an extruder (Lipex Biomembranes, Inc.) at 80° C. and pressurized with argon to 750 PSI. Vesicle size was determined by dynamic light scattering (Brookhaven Instruments). Polymerization of diacetylene lipids was achieved by cooling the vesicles to ˜2-10° C. in a 10×1 polystyrene dish (VWR) and irradiating using a hand-held UV illuminator at approximately 3.8 mW/cm² to give vesicles with a diameter of 65 nm.

B. Procedure for the preparation of liposomes. Liposomes were prepared exactly as described in EXAMPLE 1a, except the vesicles were not polymerized with UV light.

### Example 2

### Procedures for Preparing Antibody-dextran-vesicle and Antibody-vesicle Conjugates

A. Coating the polymerized vesicles: Polymerized vesicles (PVs) prepared with 95 mole percent 1,2-bis(10,12-tricosadiynoyl)-sn-glycero-phosphocholine, BisT-PC 1 (Avanti Polar Lipids) and 5 mole percent of the DTPA lipid derivative N,N-Bis[[[[(13′15′-pentacosadiynamido-3,6-dioxaoctyl)carbamoyl]methyl](carboxymethyl)amino]ethyl]-glycine 2 (Journal of the American Chemical Society (1995), 117, pp7301-7306) were coated with aminodextran as follows: PVs (10 ml, 250 mg) were added dropwise to stirred aminodextran (amine modified 10,000 MW dextran, Molecular Probes, product D-1860, 500 mg, 3 amino groups per dextran polymer) in 5 ml of 50 mM HEPES buffer at pH 8. EDAC (Aldrich 16146-2, ethyldimethylaminodipropyl carbodimimide HCl salt, 6 mg) in 200 μl water was added dropwise to the coating mixture while stirring. The mixture was stirred at room temperature overnight. The clear reaction mixture was purified by size exclusion chromatography on a Sepharose CL 4B column (2.5×30 cm, Amersham Pharmacia Biotech AB product 17-0150-01) equilibrated with 10 mM HEPES containing 200 mM NaCl at pH 7.4. When the coated PVs began to elute, 4 ml fractions were collected. The peak fractions (2 thru 6) were pooled and filtered through a 0.45μ filter (Nalgene 25 mm syringe filter, product 190-2545) followed by a 0.2μ filter (Nalgene 25 mm syringe filter, product 190-2520). The concentration of coated PV was determined by drying a sample to constant weight in an oveh maintained at 90° C.

B. Succinylation of aminodextran coated-polymerized vesicles: Aminodextran-PVs from Example 2A (15 ml, 465 mg) in 10 mM HEPES buffer at pH 7.4 were diluted with an equal volume of 200 mM HEPES buffer and the pH was adjusted to 8 with 1 N NaOH. Succinic anhydride (Aldrich product 23,969-0, 278 mg) was dissolved in 1 ml DMSO (dimethyl sulfoxide (Aldrich product 27685-5) and 100 μl aliquots were added to the coated-PV suspension with rapid stirring. The pH was monitored and adjusted as necessary to maintain the pH between 7.5 and 8 by the addition of 1 N NaOH. After the final addition of succinic anhydride, the mixture was stirred for 1 hour at room temperature and then transferred to dialysis cassettes and dialyzed against 10 mM HEPES buffer at pH 7.4.

C. Coupling of antibody to dextran-coated PVs: Succinylated dextran-vesicle conjugates from Example 2B (20 ml, 192 mg in 50 mM borate buffer at pH 8) and antibodies such as LM609, Vitaxin, and antibodies against MMP2, MMP9, PDGF receptors, FGF receptor, and VEGF receptor 2 (at about 4.67 mg/ml in 10 mM phosphate containing 150 mM NaCl, pH 6.5, 1.03 ml, 4.8 mg) were rapidly mixed while vortexing. EDAC (4 mg) in 400 μl water was added with vortexing and the mixture left at room temperature overnight. The coupling reaction mixture was made 200 mM in NaCl and the mixture was stirred at room temperature for 1 hour. The mixture was purified by size exclusion chromatography on a column of Sepharose CL 4B equilibrated with 10 mM HEPES buffer containing 200 mM NaCl at pH 7.4. Fractions (4 ml) were collected and assayed for antibody by ELISA. No free unbound antibody was detected in the column fractions. PV containing fractions were pooled and dialyzed into 50 mM histidine containing 5 mM citrate at pH 7.4.

D. Preparation of dextran-liposome conjugates: Dextran-liposome conjugates were prepared as described for the preparation of antibody-dextran-polymerized vesicle conjugates. Liposomes from Example 1B were coated with aminodextran as described in Example 2A, the aminodextran-liposome conjugates were succinylated as described in 2B.

E. Preparation of antibody-polymerized vesicle conjugates: Vitaxin was attached to vesicles from 1a as described in Example 2C.

### Example 3

### Characterization of Antibody-vesicle Conjugates by ELISA

The presence of antibodies on the dextran-vesicle conjugates was verified by ELISA as described in this example. 96-well plates were coated with goat anti-human Fc (γ) antibodies (KPL) or purified αᵥβ₃ integrin at 2 μg/mL in PBS buffer overnight. The wells were washed 3 times with 300 μL of wash solution (Wallac Delfia Wash) and blocked with 200 μL of milk blocking solution (KPL) for 1 h at RT. Antibody-vesicle conjugates (50 μL) were added at a concentration of 1-100 μg/mL in 50 mM HEPES buffer at pH 7.4. Following a 1 h incubation at RT, the wells were washed 3 times. Goat anti-human Fc (γ) antibody-HRP conjugate (KPL) in milk blocking solution at 1 μg/mL was added. Following a 1 h incubation at RT, the wells were washed twice and Lumiglo chemiluminescent substrate (KPL, 50 μL) was added. After a 1 minute incubation, the signals were monitored using a Wallac Victor luminescence reader. For non-integrin recognizing antibodies, plates coated with the appropriate antibody were used to capture the antibody conjugates. For example, plates coated with anti-mouse antibodies were used to capture antibody-vesicle conjugates prepared from mouse antibodies.

### Example 4

### Colloidal Stability of Stabilized Vesicles

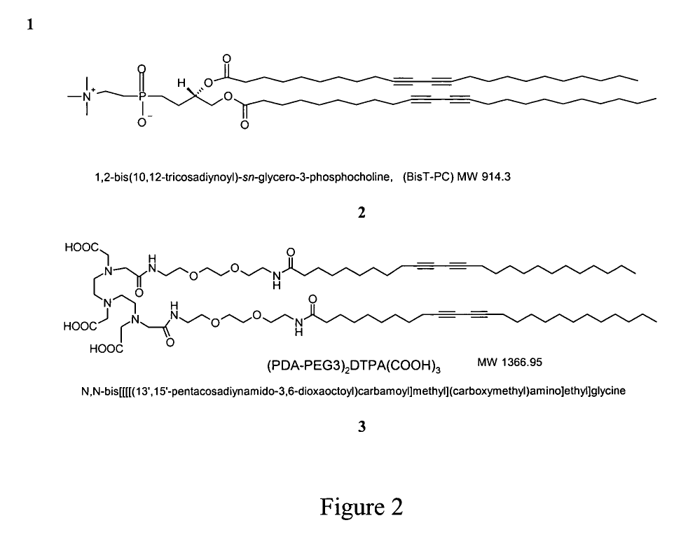

The colloidal stability of dextran-stabilized vesicles and unstabilized vesicles was compared. Each conjugate was suspended in 10 mM HEPES buffer at pH 7.4 in the absence and presence of 200 mM sodium chloride (NaCl) for 30 minutes at room temperature. FIG. 3 shows that while the mean diameter of dextran-stabilized vesicles does not change significantly in the presence of 200 mM NaCl, the size of non-coated vesicles increases 3-fold in 30 minutes.

### Example 5

### Attachment of ⁹⁰Y to Antibody-vesicle Complexes

The antibody-vesicle complex as prepared in Example 2C in 50 mM histidine buffer containing 5 mM citrate at pH 7.4 was labeled with ⁹⁰Y by diluting yttrium-90 chloride by the following procedure. Yttrium-90 chloride in 50 mM HCl (NEN Life Science Products) was diluted to a working solution containing approximately 20 mCi/ml and 100 μL was added to 5 mL of antibody-vesicle complex at 20 mg/mL in 50 mM histidine buffer containing 5 mM citrate at pH 7.4. The mixture was incubated for 30 minutes at room temperature, and the percent ⁹⁰Y bound was determined as described in Example 1.

To 100 μL of the Vitaxin-dextran-vesicles from example 2C (0.1-50 mg/mL), approximately 100-250 μCi of yttrium-90 chloride (NEN Life Science Products) was added, mixed using a vortex mixer, and incubated at room temperature for 30 minutes. In duplicate, the percent ⁹⁰Y bound to the therapeutic vesicle was determined by adding 100 μL of the ⁹⁰Y-vesicle complex to a 100k MWCO Nanosep™ (Pall Filtron) filter. The filter assembly was spun in a microfuge at 4000 rpm for 1 hr or until all of the solution has passed through the filter. The “total ⁹⁰Y” in the assembly was determined with the Capintec CRC-15R dosimeter. The filter portion of the assembly was removed and discarded. Using the dosimeter, the remaining part of the assembly containing the “unbound ⁹⁰Y” that passed through the filter was counted. “Bound ⁹⁰Y” was determined by subtracting the “unbound ⁹⁰Y” from the “total ⁹⁰Y”. Percent⁹⁰Y bound was determined by dividing the “bound ⁹⁰Y” by the “total ⁹⁰Y” and multiplying by 100. ⁹⁰Y binding was found to be greater than 75%.

### Example 6

### In vitro Comparison of Stability of Integrin-targeted Vesicle-⁹⁰Y Conjugates

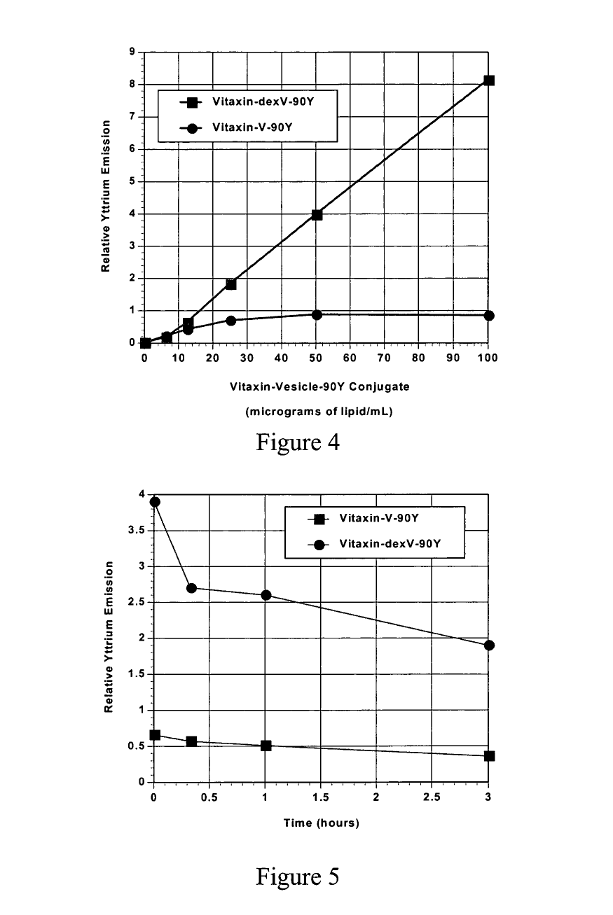

Briefly, 96 well plates coated with the αᵥβ₃ integrin (Chemicon International, Inc.) were blocked with BSA. Vitaxin-polymerized vesicle-yttrium-90 conjugates (Example 2E, or corresponding Vitaxin-dextran-liposome-yttrium-90 conjugates (Example 2C were incubated in rabbit serum for 0-3 h. Samples of rabbit serum containing 0-100 micrograms/mL of the Vitaxin-vesicle-⁹⁰Y conjugates were added and incubated for 1 hour at room temperature. The plate was washed three times with PBST buffer and the yttrium-90 was measured using a Microbeta scintillation counter (Wallac). As shown in FIG. 5, dextran-stabilized conjugates retain 7- to 6-fold more ⁹⁰Y than do the unstabilized conjugates.

### Example 7

### In vitro Comparison of ⁹⁰Y-delivery of Integrin-targeted Vesicle-⁹⁰Y Conjugates

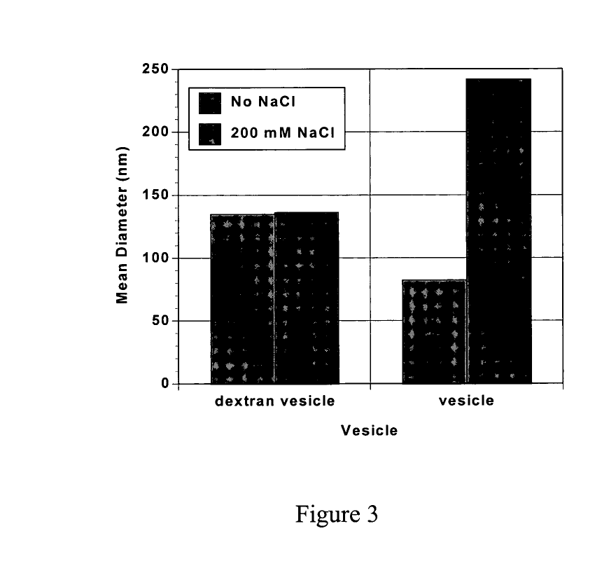

Targeting was demonstrated in-vitro using a radiometric binding assay specific to the αᵥβ₃ integrin that requires an intact tripartite complex consisting of antibody or other integrin-targeting ligand, vesicle, and yttrium-90. The dextran-stabilized Vitaxin conjugates and unstabilized Vitaxin conjugates as described in Example 6 were used in this study. For this study, ⁹⁰Y loadings were identical and comparisons were performed in at identical lipid concentrations. Antibody loadings were 4 and 6 μg of antibody/mg of lipid for the regular and dextran-stabilized liposomes, respectively. Delivery of ⁹⁰Y for the dextran-stabilized conjuagates was up to 8-fold higher than for the unstabilized conjugate, as shown in FIG. 4.

### Example 8

### Preparation of Antibody-PEI-vesicle Conjugates

A solution polyethylamine imine (PEI, 70 k molecular weight) at 100 mg/ml in 50 mM HEPES was prepared by dissolving 3 grams PEI in ˜20 ml 50 mM HEPES, adjusting the pH to 7.3 with concentrated HCl, and diluting to a final volume of 30 ml with additional buffer. PVs (20 ml, 0.5 gram) were added to PEI (15 ml, 1.5 gram) while vortexing. EDAC (50 mg) in 2 ml water was added dropwise. The mixture was left stirring at room temperature overnight. The excess PEI was removed by tangential flow filtration using 10 mM HEPES containing 200 mM NaCl pH 7.4 (1 liter) followed by 10 mM HEPES pH 7.4 (300 ml). The suspension was concentrated to 25 ml. Succinylation of the PEI-vesicle conjugates was achieved as follows. 2 ml of 0.5 M HEPES buffer at pH 7.4 was added to 20 ml PV-PEI (˜20 mg/ml, 400 mg total) and the pH adjusted to 8 with 1 N NaOH. 150 mg succinic anhydride was dissolved in 0.5 ml dry DMSO. A 50 μl aliquot of the succinic anhydride was added to the PV-PEI suspension while stirring magnetically. The pH dropped to 7.85 and was adjusted back to 8 with a few drops of 1 N NaOH. A second aliquot of succinic anhydride was added and the pH adjusted back to 8. This procedure was repeated until all of the succinic anhydride had been added. The succinylated PV-PEI was purified by continuous tangential flow filtration. Antibody coupling was performed as described in example 2C and the presence of antibody on the antibody-PEI-vesicle conjugates was determined using the procedure described in Example 3.

### Example 9

### Administration of Antibody-dextran-vesicle Complex

Rabbits that have been selected for treatment will be immobilized using a rabbit restrainer and the ear prepared with alcohol (70% isopropyl) for intravenous administration of test samples via the marginal ear vein. A 22-gauge catheter may be used for ease of test article administration. Test samples containing antibody-dextran-vesicle complex or test samples containing this complex that are labeled with ⁹⁰Y are properly drawn in sterile syringes and injected using a small needle (22-24 gauge). Intravenous injection is performed at a rate of no greater than 0.2 cc/sec. Upon delivery, gauze will be applied with pressure to minimize bleeding.

### Example 10

### Treatment of Solid Tumors in a Mouse Melanoma Model

K1735-M2 (Li et al, Invasion Metastasis (1998), 18, 1-14) tumor cells were grown in tissue culture flasks in Dubelco's medium with 10% fetal calf serum. Cells were harvested using Trypsin-EDTA solution (containing 0.05% trypsin), resuspended in PBS at 10,000,000/ml, and kept on ice. The mice were anesthetized with Nebutal (70 mg/kg). The back was shaved and prepared with alcohol solution. K1735-M2 melanoma cells were implanted by subcutaneous injection on the back with a 27-gague needle. Approximately one million cells per mouse were injected. Mice were returned to their cages when fully awake and ambulatory. Each mouse was monitored daily. Signs of abnormal behavior or poor health were recorded. Abnormal conditions were reported to the study director for appropriate care. Tumor size was measured three times a week. Animals in the study were checked daily. Animals that appeared moribund or experiencing undue stress were humanely euthanized in a CO₂ chamber. Animals with tumors were selected for treatment with the following criteria: tumors were growing and between 100 and 200 mm³. Mice were weighed on the day of treatment and 1 week after treatment. Animals wieghing greater or less than 20% the mean weight of all the animals on the day of treatment were removed from the study. Animals were treated with a single i.v. injection (approximately 200 μL per mouse) as summarized in Table 1. Hist/Cit Buffer contains 50 mM histidine and 5 mM citrate at pH 7. Other samples include the anti-mouse VEGFR-2 antibody, a conjugate consisting of this antibody and the succinylated, dextran-coated polymerized vesicles described above (anti-VEGFR-2 antibody-dexPV) as well as an antibody conjugate containing yttrium-90 (anti-VEGFR-2 antibody-dexPV-Y90), a conjugate consisting of the dextran-coated polymerized vesicle and yttrium-90 (dexPV-Y90), and a conjugate consisting of the antibody, polymerized vesicle, and yttrium-90 (anti-VEGFR-2 antibody-PV-Y90).

TABLE 1
Doses for therapeutic agents targeted to VEGFR-2 and controls

| | | Antibody | PV | Y90 | |
|---|---|---|---|---|---|
| | | Dose | Dose | Dose | # of |
| Group | Sample | (μg/g) | (mg/g) | (μCi/g) | mice |
| 1 | Hist/Cit Buffer | NA | NA | NA | 9 |
| 2 | anti-VEGFR2 Antibody | 1 | NA | NA | 9 |
| 3 | anti-VEGFR2 Antibody- | 0.8 | 0.1 | NA | 9 |
| | dexPV | | | | |
| 4 | dexPV-Y90 | NA | 0.1 | 5 | 9 |
| 5 | anti-VEGFR2-Antibody- | 0.8 | 0.1 | 5 | 9 |
| | dexPV-Y90 | | | | |
| 6 | anti-VEGFR2-Antibody- | 2 | 0.1 | 5 | 9 |
| | PV-Y90 | | | | |

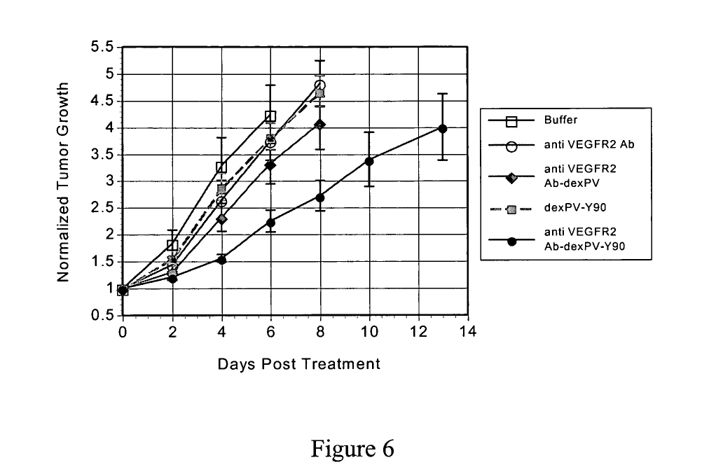

FIG. 6 and Table 2 shows the results of the experiment.

TABLE 2
Statistical analysis of tumor growth data at Day 6 with
Tukey's W procedure (P-values).^{a}

| Group | Buffer | anti VEGFR2 Ab | dexPV-Y90 |
|---|---|---|---|
| anti VEGFR2 Ab | >0.05 | N/A | N/A |
| dexPV-Y90 | >0.05 | >0.05 | N/A |
| anti VEGFR2 Ab-dexPV | >0.05 | >0.05 | >0.05 |
| anti VEGFR2 Ab-dexPV-Y90 | 0.003 | 0.043 | 0.029 |

^{a}Statistical analysis of tumor growth data at Day 6 with Tukey's W procedure. Comparison of groups with P-values less than 0.05 show statistical significance. Thus, the effect of anti VEGFR2 Ab-dexPV-Y90 in reducing tumor growth is statistically significant.

Treatment of melanoma in a murine tumor model was demonstrated with antibody-dextran-polymerized vesicle conjugates relative to controls. FIG. 6 shows treatment with anti-VEGFR2 antibody (Ab), anti-VEGFR2 Ab-dextran-polymerized vesicle conjugates (anti-VEGFR2-dexPV), dextran-polymerized vesicle-yttrium-90 complexes (dexPV-Y90), and anti-VEGFR2 Ab-dextran-polymerized vesicle-yttrium-90 complexes (anti-VEGFR2-dexPV-Y90).

A similar regimen was undertaken with other antibody-dextran-polymerized vesicle-yttrium-90 conjugates (Ab-dexPV-Y90) containing antibodies that recognize MMP2, MMP9, PDGFR A (PDGFR α), PDGFR B (PDGFR β), bFGFR, and VEGFR2. A comparison of result is shown in FIG. 7.

What is claimed is:

1. A stabilized lipid construct comprising a liposome or polymerized vesicle, a targeting entity, a therapeutic entity, and a stabilizing entity.

2. The stabilized lipid construct of claim 1, wherein the polymerized vesicle comprises 1,2-bis(10,12-tricosadiynoyl)-sn-glycero-3-phosphocholine.

3. The stabilized lipid construct of claim 1, wherein the liposome or polymerized vesicle comprises DTPA lipid derivative N,N-Bis[[[[(13′15′-pentacosadiynamido-3,6-dioxaoctyl)carbamoyl]methyl](carboxymethyl)amino]ethyl]-glycine.

4. The stabilized lipid construct of claim 1, wherein the liposome or polymerized vesicle comprises a mixture of 1,2-bis(10,12-tricosadiynoyl)-sn-glycero-3-phosphocholine and DTPA lipid derivative N,N-Bis[[[[(13′15′-pentacosadiynamido-3,6-dioxaoctyl)carbamoyl]methyl](carboxymethyl)amino]ethyl]-glycine.

5. The stabilized lipid construct of claim 1, wherein the stabilizing entity is selected from the group consisting of a natural polymer, a semi-synthetic polymer, and a synthetic polymer.

6. The stabilized lipid construct of claim 5, wherein the stabilizing entity is selected from the group consisting of dextran, modified dextran, and poly (ethylene imine).

7. The stabilized lipid construct of claim 1, wherein the stabilizing entity provides physical stability or colloidal stability.

8. The stabilized lipid construct of claim 1, wherein the stabilizing entity provides the capacity for multivalency.

9. The stabilized lipid construct of claim 1, wherein the therapeutic entity is selected from the group consisting of Y-90, Bi-213, At-211, Cu-67, Sc-47, Ga-67, Rh-105, Pr-142, Nd-147, Pm-151, Sm-153, Ho-166, Gd-159, Th-161, Eu-152, Er-171, Re-186, and Re-188.

10. The stabilized lipid construct of claim 9, wherein said therapeutic entity is ⁹⁰Y.

11. The stabilized lipid construct of claim 1, wherein said targeting entity targets the stabilized lipid construct to a cell surface.

12. The stabilized lipid construct of claim 1, wherein the targeting entity is associated with the stabilized lipid construct by covalent means.

13. The stabilized lipid construct of claim 1, wherein the targeting entity is associated with the stabilized lipid construct by non-covalent means.

14. The stabilized lipid construct of claim 1, wherein said targeting entity is an antibody.

15. The stabilized lipid construct of claim 14, wherein said antibody has a target selected from the group consisting of P-selectin, E-selectin, pleiotropin, G-protein coupled receptors, endosialin, endoglin, VEGF receptors, PDGF receptor, EGF receptor, FGF receptors, the matrix metalloproteases including MMP2 and MMP9, and prostate specific membrane antigen (PSMA).

16. The stabilized lipid construct of claim 1, wherein said targeting entity has a vascular target.

17. The stabilized lipid construct of claim 16, wherein said targeting entity is Vitaxin or LM609.

18. The stabilized lipid construct of claim 16, wherein said targeting entity is selected from the group consisting of an anti-VCAM-1 antibody, an anti-ICAM-1 antibody, an anti-VEGFR antibody, and an anti-integrin antibody.

19. A stabilized lipid construct comprising a liposome or polymerized vesicle, a therapeutic entity, and a stabilizing entity.

20. The stabilized lipid construct of claim 19, wherein the polymerized vesicle comprises 1,2-bis(10,12-tricosadiynoyl)-sn-glycero-3-phosphocholine.

21. The stabilized lipid construct of claim 19, wherein the liposome or polymerized vesicle comprises DTPA lipid derivative N,N-Bis[[[[(13′15′-pentacosadiynamido-3,6-dioxaoctyl)carbamoyl]methyl](carboxymethyl)amino]ethyl]-glycine.

22. The stabilized lipid construct of claim 19, wherein the liposome or polymerized vesicle comprises a mixture of 1,2-bis(10,12-tricosadiynoyl)-sn-glycero-3-phosphocholine and DTPA lipid derivative N,N-Bis[[[[(13′15′-pentacosadiynamido-3,6-dioxaoctyl)carbamoyl]methyl](carboxymethyl)amino]ethyl]-glycine.

23. The stabilized lipid construct of claim 19, wherein the stabilizing entity is selected from the group consisting of a natural polymer, a semi-synthetic polymer, and a synthetic polymer.

24. The stabilized lipid construct of claim 23, wherein the stabilizing entity is selected from the group consisting of dextran, modified dextran, and poly (ethylene imine).

25. The stabilized lipid construct of claim 19, wherein the stabilizing entity provides physical stability or colloidal stability.

26. The stabilized lipid construct of claim 19, wherein the stabilizing entity provides the capacity for multivalency.

27. The stabilized lipid construct of claim 19, wherein the stabilizing entity is selected from the group consisting of dextran, aminodextran and poly (ethylene imine), and wherein the targeting entity is selected from the group consisting of an anti-VCAM-1 antibody, an anti-ICAM-1 antibody, an anti-VEGFR antibody, and an anti-integrin antibody.

28. A stabilized lipid construct for controlled release of a therapeutic agent, comprising a liposome or polymerized vesicle, a therapeutic entity, and a stabilizing entity.

29. A method of treating a patient comprising administering a therapeutic agent to a patient in need thereof in a sufficient amount, said therapeutic agent comprising a stabilized lipid construct, said stabilized lipid construct comprising a liposome or polymerized vesicle, a targeting entity, a therapeutic entity, and a stabilizing entity.
